SEQUENCE LISTING (115 sequences)

SEQ ID NO: 1 - protein, 17 aa, Homo sapiens
RTHSVGYGYHLVIFTRV

SEQ ID NO: 2 - protein, 13 aa, Homo sapiens
QETLDLDAGVSEH

SEQ ID NO: 3 - protein, 12 aa, Homo sapiens
SEQETLDYWKCT

SEQ ID NO: 4 - protein, 13 aa, Homo sapiens
MKEVLDAGVSEHS

SEQ ID NO: 5 - protein, 12 aa, Homo sapiens
ETLVRQESEDYS

SEQ ID NO: 6 - protein, 12 aa, Homo sapiens
KMVSKFLTMAVP

SEQ ID NO: 7 - protein, 10 aa, Homo sapiens
SPGCISPQPA

SEQ ID NO: 8 - protein, 13 aa, Homo sapiens
HMLTHTDSQSDAG

SEQ ID NO: 9 - protein, 14 aa, Homo sapiens
HKKLYTGLPPVPGA

SEQ ID NO: 10 - protein, 14 aa, Homo sapiens
PRFPAISRFMGPAS

SEQ ID NO: 11 - protein, 15 aa, Homo sapiens
APLPTAPGRKRRVLF

SEQ ID NO: 12 - protein, 13 aa, Homo sapiens
APLPSAPRRKRRV

SEQ ID NO: 13 - protein, 13 aa, Homo sapiens
AGGRSSPGRLSRR

SEQ ID NO: 14 - protein, 13 aa, Homo sapiens
HRYKMKRQAKDKA

SEQ ID NO: 15 - protein, 16 aa, Homo sapiens
AHPGHQPGSAGQSPDL

SEQ ID NO: 16 - protein, 13 aa, Homo sapiens
KRSLASHLSGYIP

SEQ ID NO: 17 - protein, 14 aa, Homo sapiens
EKREFGLSSQWIYP

SEQ ID NO: 18 - protein, 15 aa, Homo sapiens
VTPARRRTSLPAPLS

SEQ ID NO: 19 - protein, 13 aa, Homo sapiens
SPVAASVNTTPDK

SEQ ID NO: 20 - protein, 14 aa, Homo sapiens
SPVAATPASVNTTP

SEQ ID NO: 21 - protein, 14 aa, Homo sapiens
KESPAVPPEGASAG

SEQ ID NO: 22 - protein, 14 aa, Homo sapiens
KEASPLPAESASAG

SEQ ID NO: 23 - protein, 12 aa, Homo sapiens
GHPQNLKDSELV

SEQ ID NO: 24 - protein, 12 aa, Homo sapiens
MNAEEBSLRNGG

SEQ ID NO: 25 - protein, 12 aa, Homo sapiens
MRCKRRLNSSGF

SEQ ID NO: 26 - protein, 12 aa, Homo sapiens
CKRLLFRRMYDR

SEQ ID NO: 27 - DNA, 21 bp, Homo sapiens
acaacttctccagtatcccct

SEQ ID NO: 28 - DNA, 20 bp, Homo sapiens
gcgtgaccttgacgaagatg

SEQ ID NO: 29 - DNA, 1160 bp, Homo sapiens
atgggtaacaacttctccagtatcccctcgctgccccgaggaaacccgagccgcgcgccgcggggccacccccagaacctcaaagatagcgagctggtgctcccggactgtctgcggccgcgctccttcaccgccctgcggcggccgtcgctgcggcgcgaggcggacgacgcgcgcctctcggtgagcctatgcgacctcaacgtgccgggcgcggacggcgacgaggccgcgccggccgccggctgccccatcccgcagaactcactcaactcgcagcacagccgcgcgctgccggcgcagctcgacggcgacctgcgtttccacgccctgcgcgccggcgcgcacgtccgcatcctcgacgagcagacggtggcgcgcgtggagcacgggcgcgacgagcgcgcgctcgtcttcaccagccggcccgtgcgcgtggccgagaccatcttcgtcaaggtcacgcgctcgggtggcgcgcggcccggcgcgctgtcgttcggcgtcaccacgtgcgaccccggcacgctgcggccggccgacctgcctttcagccctgaggccctggtggaccgcaaggaattctgggccgtgtgccgcgtgcccgggcccctgcacagcggcgacatcctgggcctggtggtcaacgccgacggcgagctgcacctcagccacaatggcgcggccgccggcatgcagctgtgcgtggacgcctcgcagccgctttggatgctcttcggcctgcacgggaccatcacgcagatccgcatcctcggctccactatcctggccgagcggggtatcccgtcactcccctgctcccctgcctccacgccaacctcgcccagtgccctgggcagccgcctgtctgacccttgctcagcacgtgcagctctggccctctgggtagctctgctggtgggacagcccccaattcgccagtgagcctgcccgagtcgccagtgaccccaggtctgggccagtggagcgatgagtgcaccatttgctatgaacacgcggtggacacggtcatctacacatgtggccacatgtgcctctgctacgcctgtggcctgcgcctcaagaaggctctgcacgcctgctgccccatctgccgccgccccatcaaggacatcatcaagacctaccgcagctcctag

SEQ ID NO: 30 - protein, 386 aa, Homo sapiens
MGNNFSSIPSLPRGNPSRARRGHPQNLKDSELVLPDCLRPRSFTALRRPSLRREADDARLSVSLCDLNVPGADGDEAAPAAGCPIPQNSLNSQHSRALPAQLDGDLRFHALRAGAHVRILDEQTVARVEHGRDERALVFTSRPVRVAETIFVKVTRSGGARPGALSFGVTTCDPGTLRPADLPFSPEALVDRKEFWAVCRVPGPLHSGDILGLVVNADGELHLSHNGAAAGMQLCVDASQPLWMLFGLHGTITQIRILGSTILAERGIPSLPCSPASTPTSPSALGSRLSDPLLSTCSSGPLGSSAGGTAPNSPVSLPESPVTPGLGQWSDECTICYEHAVDTVIYTCGHMCLCYACGLRLKKALHACCPICRRPIKDIIKTYRSS

SEQ ID NO: 31 - DNA, 20 bp, Homo sapiens
agggttatgagactatactg

SEQ ID NO: 32 - DNA, 19 bp, Homo sapiens
ggtggtgggttgggataag

SEQ ID NO: 33 - DNA, 1128 bp, Homo sapiens
ccagggttatgagactatcactgctcaggacctactaacaataaaggaaatcgaaacatgaccaaatcgtacagcgagagtgggctgatgggcgagcctcagccccaaggtcctccaagctggacagacgagtgtctcagttctcaggacgaggagcacgaggcagacaagaaggaggacgacctcgaagccatgaacgcagaggaggactcactgaggaacgggggagaggaggaggacgaagatgaggacctggaagaggaggaagaagaggaagaggaggatgacgatcaaaagcccaagagacgcggccccaaaaagaagaagatgactaaggctcgcctggagcgttttaaattgagacgcatgaaggctaacgcccgggagcggaaccgcatgcacggactgaacgcggcgctagacaacctgcgcaaggtggtgccttgctattctaagacgcagaagctgtccaaaatcgagactctgcgcttggccaagaactacatctgggctctgtcggagatcctgcgctcaggcaaaagcccagacctggtctccttcgttcagacgctttgcaagggcttatcccaacccaccaccaacctggttgggggctgcctgcaactcaatcctcggacttttctgcctgagcagaaccaggacatgcccccccacctgccgacggccagcgcttccttccctgtacacccctactcctaccagtcgcctgggctgcccagtccgccttacggtaccatggacagctcccatgtcttccacgttaagcctccgccgcacgcctacagcgcagcgctggagcccttctttgaaagccctctgactgattgcaccagcccttcctttgatggacccctcagcccgccgctcagcatcaatggcaacttctctttcaaacacgaaccgtccgccgagtttgagaaaaattatgcctttaccatgcactatcctgcagcgacactggcaggggcccaaagccacggatcaatcttctcaggcaccgctgcccctcgctgcgagatccccatagacaatattatgtccttcgatagccattcacatcatgagcgagtcatgagtgcccagctcaatgccatatttcatgattag

SEQ ID NO: 34 - protein, 12 aa, Homo sapiens
PRVMRLSLLRTY

SEQ ID NO: 35 - protein, 329 aa, Homo sapiens
SSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVGGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 36 - DNA, 20 bp, Homo sapiens
acggtccacactcacctctc

SEQ ID NO: 37 - DNA, 20 bp, Homo sapiens
ggttcaacacggtctggttt

SEQ ID NO: 38 - DNA, 1254 bp, Homo sapiens
atggcagtggagaccacggtccacactcacctctctgcgtctccaccgcagggctctccctacgaccacacacccggcatggcgggctccttggggtaccatccttacgcggcgcccctgggatcgtacccttacggggacccagcgtaccggaagaacgccacaagggacgccacggctaccctcaaggcctggctcaacgagcaccgcaagaacccctaccccaccaagggcgagaagatcatgctggccatcatcaccaagatgaccctcacccaggtgtccacctggttcgccaacgcgcgccggcgcctcaagaaagagaataaaatgacgtggacgccgcggaaccgcagcgaggacgaggaagaggaggagaacattgacctggagaagaacgacgaggacgagccccagaagcccgaggacaagggcgaccccgagggccccgaagcaggaggagctgagcagaaggcggcttcgggctgcgaacggcttcagggaccacccacccctgcaggcaaggagacggagggcagcctcagcgactcggattttaaggagccgccctcggagggccgcctcgacgcgctgcagggccccccccgcaccggcgggccctccccggctgggccagcggcggcgcggctggcggaggacccggcccctcactaccccgccggagcgccggcgcccggcccgcatccagccgcgggcgaggtgcctccgggtcccggcgggccctcggttatccattcgccgcctccgccgccgcctcctgcggtgctcgccaagcccaaactgtggtctttggcagagatcgccacattgtcggacaaggtcaaggacgggggcggcgggaacgagggctctccatgcccaccgtgtcccgggcccatagccgggcaagccctaggaggcagccgggcgtcgccggccccggcgccgtcacgctcgccctcggcgcagtgtccttttccaggcgggacggtgctgtcccggcctctctactacaccgcgcccttctatcccggctacacgaactatggctccttcggacaccttcatggccacccggggcccgggccaggccccacaaccggtccggggtctcatttcaatggattaaaccagaccgtgttgaaccgagcggacgctttggctaaagacccgaaaatgttgcggagccagtctcagctagacctgtgcaaagactctccctatgaattgaagaaaggtatgtccgacatttaa

SEQ ID NO: 39 - DNA, 20 bp, Homo sapiens
agtctcccggggattttgta

SEQ ID NO: 40 - DNA, 20 bp, Homo sapiens
ccgacgagtaggatgagacc

SEQ ID NO: 41 - DNA, 930 bp, Homo sapiens
ggaggagaaaaagcattttcactttttttgctcccactctaagaagtctcccggggattttgtatatattttttaacttccgtcagggctcccgcttcatatttccttttctttccctctctgttcctgcacccaagttctctctgtgtccccctcgcgggccccgcacctcgcgtcccggatcgctctgattccgcgactccttggccgccgctgcgcatggaaagctctgccaagatggagagcggcggcgccggccagcagccccagccgcagccccagcagcccttcctgccgcccgcagcctgtttctttgccacggccgcagccgcggcggccgcagccgccgcagcggcagcgcagagcgcgcagcagcagcagcagcagcagcagcagcagcagcaggcgccgcagctgagaccggcggccgacggccagccctcagggggcggtcacaagtcagcgcccaagcaagtcaagcgacagcgctcgtcttcgcccgaactgatgcgctgcaaacgccggctcaacttcagcggctttggctacagcctgccgcagcagcagccggccgccgtggcgcgccgcaacgagcgcgagcgcaaccgcgtcaagttggtcaacctgggctttgccacccttcgggagcacgtccccaacggcgcggccaacaagaagatgagtaaggtggagacactgcgctcggcggtcgagtacatccgcgcgctgcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttctga

SEQ ID NO: 42 - protein, 223 aa, Homo sapiens
MESSAKMESGGAGQQPQPQPQQPFLPPAACFFATQSAQQQQQQQQQQQQAPQLRPAADGQPSGGGHKSAPKQVKRQRSSSPELMRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNWF

SEQ ID NO: 43 - protein, 12 aa, Homo sapiens
GHPQNLKDSELV

SEQ ID NO: 44 - protein, 12 aa, Homo sapiens
MNAEEBSLRNGG

SEQ ID NO: 45 - protein, 12 aa, Homo sapiens
MRCKRRLNSSGF

SEQ ID NO: 46 - protein, 12 aa, Homo sapiens
CKRLLFRRMYDR

SEQ ID NO: 47 - DNA, 24 bp, Homo sapiens
ccctctctgttcctgcacccaagt

SEQ ID NO: 48 - DNA, 26 bp, Homo sapiens
ccagttggtgaagtcgagaagctcct

SEQ ID NO: 49 - DNA, 24 bp, Homo sapiens
cggtccttgcgccaggtcctttga

SEQ ID NO: 50 - DNA, 26 bp, Homo sapiens
gtactagcgacacccacaaccctcca

SEQ ID NO: 51 - DNA, 26 bp, Homo sapiens
accggtcgttccgatggcagtggaga

SEQ ID NO: 52 - DNA, 23 bp, Homo sapiens
cgcgttaaatgtcggacatacct

SEQ ID NO: 53 - DNA, 26 bp, Homo sapiens
ccaccatgggtaacaacttctccagt

SEQ ID NO: 54 - DNA, 25 bp, Homo sapiens
gctaggagctgcggtaggtcttgat

SEQ ID NO: 55 - DNA, 25 bp, Homo sapiens
gccccagggttatgagactatcact

SEQ ID NO: 56 - DNA, 25 bp, Homo sapiens
ggtgaaactggcgtgcctctaatca

SEQ ID NO: 57 - DNA, 26 bp, Homo sapiens
gactccaggagacgatgcgacactca

SEQ ID NO: 58 - DNA, 26 bp, Homo sapiens
gacaggggaggtgaatgaccactgtt

SEQ ID NO: 59 - DNA, 25 bp, Homo sapiens
cagtgatctggaggagctggagcaa

SEQ ID NO: 60 - DNA, 27 bp, Homo sapiens
ggcgatcagcaggatctcctctgaggt

SEQ ID NO: 61 - DNA, 29 bp, Homo sapiens
ccagcatgggtagcaagaaactaaaacga

SEQ ID NO: 62 - DNA, 25 bp, Homo sapiens
ctgtctctaggaatttccataggct

SEQ ID NO: 63 - DNA, 26 bp, Homo sapiens
gcaagatgtctgggactgaggaagca

SEQ ID NO: 64 - DNA, 26 bp, Homo sapiens
gtggcctgagcctcagtaagatggat

SEQ ID NO: 65 - DNA, 28 bp, Homo sapiens
ccacgatggtgccctccagcccagcggt

SEQ ID NO: 66 - DNA, 24 bp, Homo sapiens
gcccgagagtcactggttcacatt

SEQ ID NO: 67 - DNA, 25 bp, Homo sapiens
gtgaccgctgcggctacaatactaa

SEQ ID NO: 68 - DNA, 25 bp, Homo sapiens
ggacaagtaggatgcttagatttga

SEQ ID NO: 69 - DNA, 29 bp, Homo sapiens
cgtatgttcaggtccaaacgctcggggct

SEQ ID NO: 70 - DNA, 26 bp, Homo sapiens
ccgccactatctggggttgttgagga

SEQ ID NO: 71 - protein, 14 aa, Homo sapiens
AQQASPGCISPQPA

SEQ ID NO: 72 - DNA, 20 bp, Homo sapiens
cacctgaaacaccacaccag

SEQ ID NO: 73 - DNA, 20 bp, Homo sapiens
gcccattgtgaacctgtctt

SEQ ID NO: 74 - DNA, 20 bp, Homo sapiens
gagcaggcaaatgtgcaata

SEQ ID NO: 75 - DNA, 24 bp, Homo sapiens
tctgagagttcttgtccttcttca

SEQ ID NO: 76 - DNA, 20 bp, Homo sapiens
gagcaggcaaatgtgcaata

SEQ ID NO: 77 - DNA, 20 bp, Homo sapiens
tgttgcaatgtgatggaagg

SEQ ID NO: 78 - DNA, 20 bp, Homo sapiens
gaccctggttagaccaaagc

SEQ ID NO: 79 - DNA, 20 bp, Homo sapiens
cctgatccaaccaatcacct

SEQ ID NO: 80 - DNA, 20 bp, Homo sapiens
gagcaggcaaatgtgcaata

SEQ ID NO: 81 - DNA, 22 bp, Homo sapiens
tttttgtgcaccaacagacttt

SEQ ID NO: 82 - DNA, 20 bp, Homo sapiens
gagccagctcaagaaaatgg

SEQ ID NO: 83 - DNA, 20 bp, Homo sapiens
gacctgaataagccccaaca

SEQ ID NO: 84 - DNA, 20 bp, Homo sapiens
cgcgctgctactcacatact

SEQ ID NO: 85 - DNA, 20 bp, Homo sapiens
caaccaggtgtttgccttct

SEQ ID NO: 86 - DNA, 20 bp, Homo sapiens
gtgatgaagagcagggcagt

SEQ ID NO: 87 - DNA, 20 bp, Homo sapiens
gtcccgtgaggtgaggtcta

SEQ ID NO: 88 - DNA, 21 bp, Homo sapiens
agttcctggtatgtgggaaga

SEQ ID NO: 89 - DNA, 19 bp, Homo sapiens
tcctgtaatgtcgcaaggt

SEQ ID NO: 90 - DNA, 20 bp, Homo sapiens
aggacaccatgaggaacagc

SEQ ID NO: 91 - DNA, 20 bp, Homo sapiens
gccatgttcttgctcacgtc

SEQ ID NO: 92 - DNA, 20 bp, Homo sapiens
accaggacaccatgaggaac

SEQ ID NO: 93 - DNA, 19 bp, Homo sapiens
gggccatgttcttgctcac

SEQ ID NO: 94 - DNA, 18 bp, Homo sapiens
gagcggcatgaacatgag

SEQ ID NO: 95 - DNA, 19 bp, Homo sapiens
gtcgctccagctcgtacac

SEQ ID NO: 96 - DNA, 19 bp, Homo sapiens
gccgaatcatgtcgatgag

SEQ ID NO: 97 - DNA, 19 bp, Homo sapiens
ccctccatgcccactttct

SEQ ID NO: 98 - DNA, 19 bp, Homo sapiens
ccagcatgatccacctgac

SEQ ID NO: 99 - DNA, 18 bp, Homo sapiens
gctgagcctgttgctgct

SEQ ID NO: 100 - DNA, 19 bp, Homo sapiens
caacaggctcagcagcagt

SEQ ID NO: 101 - DNA, 20 bp, Homo sapiens
gaggagttcaggtgggacag

SEQ ID NO: 102 - DNA, 20 bp, Homo sapiens
aaaaacggagtttggcctct

SEQ ID NO: 103 - DNA, 20 bp, Homo sapiens
ctgcaactgtcgagagcatc

SEQ ID NO: 104 - DNA, 20 bp, Homo sapiens
ggcaaaacatttgcaatgaa

SEQ ID NO: 105 - DNA, 20 bp, Homo sapiens
cttgcccttgtttccttttg

SEQ ID NO: 106 - DNA, 19 bp, Homo sapiens
ccgaagtgctggagctgac

SEQ ID NO: 107 - DNA, 18 bp, Homo sapiens
gagggtgggagggagaga

SEQ ID NO: 108 - DNA, 21 bp, Homo sapiens
aaaaggaaaatgccagctgat

SEQ ID NO: 109 - DNA, 20 bp, Homo sapiens
tgctcttcgtcttttctcca

SEQ ID NO: 110 - DNA, 20 bp, Homo sapiens
aaattcctcgtccccggtag

SEQ ID NO: 111 - DNA, 20 bp, Homo sapiens
tcagctggctcagactttca

SEQ ID NO: 112 - DNA, 18 bp, Homo sapiens
gtctccagcctgcccttc

SEQ ID NO: 113 - DNA, 20 bp, Homo sapiens
ccgattggtcttgtgtttcc

SEQ ID NO: 114 - DNA, 18 bp, Homo sapiens
gtctccagcctgcccttc

SEQ ID NO: 115 - DNA, 20 bp, Homo sapiens
ctgaatttcccgacttgacc

(19) United States
(12) Patent Application Publication
(10) Pub. No.: US 2004/0219575 A1
(43) Pub. Date: Nov. 4, 2004

(54) Methods and compositions for the diagnosis, prognosis, and treatment of cancer

(76) Inventors: Toomas Neuman, Santa Monica, CA (US); Kaia Palm, Tallinn (EE)

Correspondence Address: DORSEY & WHITNEY LLP, INTELLECTUAL PROPERTY DEPARTMENT, 4 EMBARCADERO CENTER, SUITE 3400, SAN FRANCISCO, CA, 94111, US

(21) Appl. No.: 10/746,547

(22) Filed: Dec. 24, 2003

Related U.S. Application Data
(60) Provisional application No. 60/436,693, filed on Dec. 26, 2002.

(51) Int. Cl. C12Q 1/68; G01N 33/574

(52) U.S. Cl. 435/6; 435/7.23

(57) ABSTRACT

Disclosed herein are compositions and methods for diagnosing cancer, determining cancer prognosis, and identifying cancer subtypes for a variety of cancers. Also disclosed herein are compositions and methods for the treatment of cancer.

This application claims priority to U.S. provisional patent application serial No. 60/436,693, filed 26 Dec. 2002, which is expressly incorporated herein in its entirety by reference.

### FIELD

The present disclosure relates to the expression of transcription modulator splice variants, and to the early diagnosis, prognosis, and treatment of cancer. The present disclosure further relates to the molecular characterization of cancer and the description of cancer subtypes, as well as the optimization of cancer treatment. The present disclosure further relates to cancer treatment methods and therapeutic agents.

### BACKGROUND

The early and accurate detection of cancer, and the precise characterization of tumor cells are highly desirable for effective cancer treatment. However, many current diagnostic methods, such as those involving imaging and the analysis of biochemical markers, are not reliable and do not provide for early diagnosis.

A number of studies examining the molecular characteristics of various cancers have been reported. Oligonucleotide and cDNA micro-arrays (Bhattacharjee et al., Proc. Natl. Acad. Sci. USA, 98(24):13790-13795 (2001), Garber et al., Proc. Natl. Acad. Sci. USA 98(24):13784-13789 (2001), Virtanen et al., Proc. Natl. Acad. Sci. USA, 99(19):12357-12362 (2002)), as well as the serial analysis of gene expression (Nacht et al., Proc. Natl. Acad. Sci. USA, 98(26):15203-15208 (2001)) have been used to molecularly characterize different cancer types. In addition, the expression of particular markers has been associated with prognosis for particular cancers (Beer et al., Nature Medicine, 8(8):816-824 (2002), Volm et al., Clinical Cancer Res., 8:1843-1848 (2002), Wigle et al., Cancer Res., 62:3005-3008 (2002)). Tumor cells have also been shown to express splice variant mRNAs that are not present in normal cells of the same cell type. A genome-wide computational screen using human expressed sequence tags identified more than 25,000 alternatively spliced transcripts, of which 845 were significantly associated with cancer (Wang et al., Cancer Research 63:655-657 (2003)).

Differences between the gene expression profiles of cancer cells and normal cells, and the presence of cancer cell markers, stem in part from differences in patterns of transcriptional activity between cancer and normal cells. It is well known that a number of identified oncogenes encode transcription factors. In addition, it has been reported that some tumor cells aberrantly express transcriptional modulators that are normally expressed during development (Palm et al., Brain Res. Mol. Brain Res. 72(1):30-39 (1999), Lee et al., J. Mol. Neurosci., 15(3):205-214 (2000), Lawinger et al., Nat. Med., 6(7):826-831 (2000), Coulson et al., Cancer Res., 60(7):1840-1844 (2000), Gure et al., Proc. Natl. Acad. Sc. USA., 97(8):4198-203.(2000)). WO 02/40716 in particular discloses the expression profiles of a number of transcription factors in a variety of cancers, and describes tumor subtypes that express subsets of transcription factors.

Studies examining the immunoreactivity of blood sera from cancer patients have also been reported. Serological analysis of expression cDNA libraries has been used to identify tumor antigens, among which developmentally regulated transcription factors have been found (Gure et al., 2000). Additionally, WO 02/40716 discloses the use of peptides derived from developmentally regulated transcription factors to generate an anti-transcription-factor autoantibody profile detailing the aberrant expression of the transcription factors in tumor cells. However, because these transcription factors are not tumor-specific and are potentially exposed to the immune system prior to the onset of cancer, the use of immunoreactivity against such transcription factors to diagnose cancer may be hindered by the occurrence of false positive results.

Despite this knowledge of the molecular characteristics of a variety of cancers, current diagnostic markers and methodologies cannot reliably distinguish between an aggressive cancer that has metastatic potential, and an indolent tumor that does not threaten patient survival. Tumor-specific or tumor-enriched molecular markers that could reliably be used to determine the presence of cancer at early stages of the disease would be of tremendous use. Further, markers that could reliably be used to characterize the molecular phenotype of a tumor cell of a particular cancer type would be of tremendous use.

### SUMMARY

The present disclosure describes the expression profiles of a plurality of transcription modulator splice variants that are tumor-specific or tumor-enriched (“tumor-specific/enriched”), and further describes their correlation with numerous cancer types and subtypes. The present disclosure further establishes that the determination of the expression of a plurality of such transcription modulator splice variants provides a very highly accurate diagnostic indicator for the early detection of cancer. Further, the determination of the expression of an appropriate set of a plurality of such transcription modulator splice variants as disclosed herein is indicative of cancer for a variety of cancer types. Combinatorial expression-determination methods disclosed herein may thus be used to diagnose a variety of cancers with very high accuracy. As further disclosed herein, determining the expression of a battery of such transcription modulator splice variants may be reliably used to identify cancer subtypes and thereby optimize treatment.

While the expression of transcription factors in a variety of cancer types has been previously reported, and the use of such expression profiles as a diagnostic tool has been disclosed in WO 02/40716, the present methods are distinguished in one respect by their reliance on the expression profiles of tumor-enriched or tumor-specific splice variants of transcription modulators, which are more specific to cancer and, in many tumor types, more highly expressed than their wildtype counterparts. The present disclosure thus provides diagnostics that are both more sensitive and more accurate than those disclosed in the prior art.

Additionally, while the expression of particular splice variants of individual transcription factors has been observed in certain cancers (e.g., Coulson et al.), the present disclosure establishes that a plurality of genes encoding transcription modulators express splice variants that are tumor-specific/enriched and associated with a variety of cancers and tumor cell types. The methods disclosed herein are further distinguished from the prior art by being focused on a plurality of such splice variants. As disclosed herein, an appropriate set of a plurality of such transcription modulator splice variants may be used to diagnose cancer with very high accuracy across cancer types.

Accordingly, disclosed herein are methods and compositions for diagnosing cancer. Further disclosed herein are methods and compositions for diagnosing cancer subtypes. Further disclosed herein are methods and compositions for determining the prognosis of a patient having cancer. Further disclosed herein are methods and compositions for the treatment of cancer.

The diagnostic methods provided herein generally comprise determining the expression of a plurality of tumor-specific/enriched splice variants of transcription modulators. Typically, the expression of at least two, more preferably at least 5, still more preferably at least 10, and often at least 15, 25 or 50 transcription modulator splice variants is determined, and generally not more than about 5000, more preferably less than about 1000 or 500, and still more preferably less than about 250 or 100 such splice variants are employed in the subject methods.

In a preferred embodiment, the expression of at least one splice variant of each of a plurality of transcription modulators is determined. In a preferred embodiment, the expression of at least one splice variant of between at least two and about 1000, more preferably between at least two and about 500, more preferably between at least two and about 250, more preferably between at least two and about 150, more preferably between at least two and about 100, more preferably between at least two and about 75, more preferably between at least two and about 50, more preferably between at least two and about 25, more preferably between at least two and about 10 transcription modulators is determined, wherein expression of each of the transcription modulator splice variants is indicative of cancer.

In another preferred embodiment, the expression of a plurality of splice variants of a transcription modulator is determined. In a preferred embodiment, the expression of between at least two and about 10 or 20, more preferably between at least two and about 5 splice variants of a transcription modulator is determined, wherein expression of each of the transcription modulator splice variants is indicative of cancer.

The expression of a plurality of transcription modulator splice variants may be determined simultaneously or sequentially

Though the expression of each of a plurality of transcription modulator splice variants is indicative of cancer, each splice variant is not necessarily expressed in all cancers, all tumor cell types, or all patients having a particular type of cancer (e.g., prostate cancer; small cell lung cancer). Further, in some embodiments, the set of transcription modulator splice variants for which expression is determined in a diagnostic assay will include one or more that are determined not to be expressed (i.e., in addition to the plurality that are determined to be expressed). As disclosed herein, it is the overall expression pattern, i.e., the combined determinations of the expression of a plurality of transcription modulator splice variants, not individual splice variants, that provides for the highly accurate diagnosis of cancer. Thus, negative expression results are obtained for individual splice variants in some diagnostic assays disclosed herein, yet the assay results are indicative of cancer (owing to the determined expression of other tumor-specific/enriched splice variants). As further disclosed herein, the absence of expression of transcription modulator splice variants in a diagnostic assay is useful for the identification of cancer subtypes.

It will be apparent to one of skill in the art that the information gleaned from the determination of the expression of a plurality of transcription modulator splice variants is, as exemplified herein, not simply additive. Rather, the combinatorial analysis of tumor-enriched/specific splice variant expression disclosed herein reveals molecular subtypes of cancer, in which the expression of a number of such splice variants is linked.

The present methods thus satisfy the need for a highly accurate diagnostic method, and provide for the precise characterization of tumor cells and the identification of cancer subtypes.

In a preferred embodiment disclosed herein are methods for diagnosing cancer subtypes. The methods generally comprise determining the expression of a plurality of tumor-specific/enriched splice variants of transcription modulators. In a preferred embodiment, the methods comprise determining the expression of at least one splice variant of a plurality of transcription modulators, wherein the presence or absence of expression of each splice variant is indicative of a cancer subtype. In another preferred embodiment, the methods comprise determining the expression of a plurality of splice variants of a transcription modulator, wherein the presence or absence of expression of each splice variant is indicative of a cancer subtype. In a preferred embodiment, the cancer subtype is characterized by its metastatic potential. In another embodiment, the cancer subtype is characterized by its refractory behavior, particularly its tolerance to a therapeutic agent. In another preferred embodiment, the cancer subtype is characterized by its invasive activity.

In a preferred embodiment, the methods further comprise determining the expression of additional markers which are not transcription modulator splice variants but which are useful markers of tumor cell subtypes. Examples of such markers include integrins, receptors for extracellular signals including receptor tyrosine kinases, non-receptor tyrosine kinases, matrix metalloproteinases, and other molecules known to have a role in signal transduction, cell proliferation, cell motility, cell adhesion, or cell survival.

In another preferred embodiment disclosed herein are methods for determining cancer prognosis, which comprise diagnosing a cancer subtype as disclosed herein. In a preferred embodiment, the methods further comprise determining the expression of additional prognostic indicators known in the art.

Determining splice variant expression may involve determining mRNA or protein expression, which may be done using any of the large number of methods known in the art. Alternatively, determining splice variant expression may involve determining the presence of autoantibodies that recognize the splice variant.

A preferred method for determining expression involves the use of RT-PCR to determine the expression of mRNAs encoding transcription modulator splice variants. Another preferred method for determining expression involves the use oligonucleotide probes to determine the expression of mRNAs encoding transcription modulator splice variants. In a particularly preferred embodiment, the oligonucleotide probes are in an array. Another preferred method for determining expression involves the use of peptides that are capable of detecting auto-antibodies that recognize transcription modulator splice variants. The peptides do not specifically bind to autoantibodies that specifically bind to wildtype isoforms of transcription modulators. In a particularly preferred embodiment, the peptides are in an array.

Importantly, the methods provided herein provide for distinguishing the expression of splice variants of transcription modulators from the expression of “wildtype” isoforms of these transcription modulators. As disclosed herein, many tumor-specific/enriched splice variants of transcription modulators have wildtype counterparts that are expressed in non-tumor cells. Consequently, distinguishing splice variant from wildtype isoform expression contributes significantly to the accuracy of the diagnostic methods disclosed herein.

Preferred transcription modulators for use in the presently disclosed methods are those having splice variant isoforms that are tumor-specific/enriched. Especially preferred transcription modulators include NRSF, MDM2, TSG, RREB, ZNF207, TTF-1, GTFIIIA, HES-6, HRY, Msx2, Neu, NeuroD1, Mash-1, Irx2.

Preferred splice variants of transcription modulators are those for which expression is indicative of cancer, particularly cancer selected from the group consisting of lung cancer (e.g., small cell lung cancer, non-small cell lung cancer), gastrointestinal cancer (e.g., colorectal cancer, stomach cancer, liver cancer, pancreatic cancer, and cancers of other regions of gastrointestinal tract), breast cancer, prostate cancer, skin cancer (e.g., basal cell carcinoma, melanoma), sarcoma, endocrine cancer (e.g., carcinoids, insulinoma, cancer of thyroid gland), neural cancers (e.g., neuroblastoma, glioblastoma, medulloblastoma, retinoblastoma), bladder cancer, cervical cancer, renal cancer, hematopoietic cancers (e.g., lymphoma, leukemia). Also preferred are splice variants for which the presence or absence of expression is indicative of a cancer subtype, particularly a subtype within a cancer selected from the group consisting of lung cancer (e.g., small cell lung cancer, non-small cell lung cancer), gastrointestinal cancer (e.g., colorectal cancer, stomach cancer, liver cancer, pancreatic cancer, and cancers of other regions of gastrointestinal tract), breast cancer, prostate cancer, skin cancer (e.g., basal cell carcinoma, melanoma), sarcoma, endocrine cancer (e.g., carcinoids, insulinoma, cancer of thyroid gland), neural cancers (e.g., neuroblastoma, glioblastoma, medulloblastoma, retinoblastoma), bladder cancer, cervical cancer, renal cancer, hematopoietic cancers (e.g., lymphoma, leukemia).

Especially preferred tumor-specific/enriched transcription modulator splice variants for use in the subject methods include those disclosed at Genbank accession numbers AF228045, NM_{—}006878, NM_{—}006879, NM_{—}006880, NM_{—}006880, AY207474, A1924329, NP 002946, A1870134, BAA23529, BAA23529, BC006221, BC006221, NM_{—}003317, NM_{—}003317, U14134, NP_{—}002088, AK075040, BC039152, AF264785, X69295, and D31771. Also especially preferred are the novel tumor-specific/enriched splice variants of Neu, NeuroD1, Mash-1, and Irx2 disclosed in FIGS. 4-7.

Preferred peptides for use in the detection of autoantibodies that recognize tumor-specific/enriched transcription modulator splice variants are those that do not specifically bind to autoantibodies that specifically bind to corresponding wildtype isoforms of transcription modulators. Especially preferred peptides include RTHSVGYGYHLVIFTRV, QETLDLDAGVSEH, SEQETLDYWKCT, MKEVLDAGVSEHS, ETLVRQESEDYS, KMVSKFLTMAVP, SPGCISPQPA, HMLTHTDSQSDAG, HKKLYTGLPPVPGA, PRFPAISRFMGPAS, APLPTAPGRKRRVLF, APLPSAPRRKRRV, AGGRSSPGRLSRR, HRYKMKRQAKDKA, AHPGHQPGSAGQSPDL. KRSLASHLSGYIP, EKREFGLSSQWIYP, VTPARRRTSLPAPLS, SPVAASVNTTPDK, SPVAATPASVNTTP, KESPAVPPEGASAG, and KEASPLPAESASAG.

Also especially preferred are the following peptides that specifically bind to autoantibodies that specifically bind to novel tumor-specific/enriched splice variants of Neu, NeuroD1, Mash-1, and Irx2, respectively: GHPQNLKDSELV, MNAEEBSLRNGG, MRCKRRLNSSGF, and CKRLLFRRMYDR.

In another preferred embodiment disclosed herein are peptide arrays, which arrays comprise a plurality of peptides derived from tumor-specific/enriched transcription modulator splice variants, wherein the peptides specifically bind to autoantibodies which are characterized by their ability to specifically bind to transcription modulator splice variants that are tumor-specific/enriched. Moreover, the peptides are splice-variant specific in that they do not bind to autoantibodies that specifically bind to wildtype isoforms of the transcription modulators. Such arrays find use in cancer diagnosis, and may particularly be used to determine the expression of a plurality of transcription modulator splice variants simultaneously. In a preferred embodiment, such peptide arrays comprise peptides that specifically bind to autoantibodies that specifically bind to novel tumor-specific/enriched splice variants of Neu, NeuroD1, Mash-1, and Irx2 disclosed herein.

In another preferred embodiment provided herein are peptide arrays that consist essentially of a plurality of peptides derived from tumor-specific/enriched transcription modulator splice variants, wherein the peptides specifically bind to autoantibodies which are characterized by their ability to specifically bind to transcription modulator splice variants that are tumor-specific/enriched. Moreover, the peptides are splice-variant specific in that they do not bind to autoantibodies that specifically bind to wildtype isoforms of the transcription modulators. In a preferred embodiment, such peptide arrays comprise peptides that specifically bind to autoantibodies that specifically bind to novel tumor-specific/enriched splice variants of Neu, NeuroD1, Mash-1, and Irx2 disclosed herein.

Also disclosed herein in a preferred embodiment are oligonucleotide arrays, which arrays comprise a plurality of oligonucleotides derived from the nucleotide sequences of mRNAs encoding tumor-specific/enriched transcription modulator splice variants, and which hybridize under high stringency conditions (0.2×SSC, 65° C.) to such mRNAs or their complements. Such arrays find use in cancer diagnosis, and may particularly be used to determine the expression of a plurality of transcription modulator splice variants simultaneously. In a preferred embodiment, such arrays comprise oligonucleotides that are substantially complementary to mRNAs encoding novel tumor-specific/enriched splice variants of Neu, NeuroD1, Mash-1, and Irx2 disclosed herein, or their complements.

In another preferred embodiment provided herein are oligonucleotide arrays that consist essentially of a plurality of such oligonucleotides derived from the nucleotide sequences of mRNAs encoding tumor-specific/enriched transcription modulator splice variants. In a preferred embodiment, such arrays comprise oligonucleotides substantially complementary to mRNAs encoding novel tumor-specific/enriched splice variants of Neu, NeuroD1, Mash-1, and Irx2 disclosed herein, or their complements.

Also disclosed herein are methods for the treatment of cancer, and therapeutics useful in the treatment of cancer.

The treatment methods generally comprise determining the expression of a plurality of tumor-specific/enriched transcription modulator splice variants, wherein the expression of each of the transcription modulator splice variants is indicative of cancer, and further comprise administering to the patient a bioactive agent capable of inhibiting the activity of one or more of such splice variants determined to be expressed. In a preferred embodiment, the methods comprise determining the expression of at least one splice variant of each of a plurality of transcription modulators. In another preferred embodiment, the methods comprise determining the expression of a plurality of splice variants of a transcription modulator. As in the methods described above, expression of tumor-specific/enriched splice variants is distinguished from the expression of corresponding wildtype isoforms of transcription modulators.

In a preferred embodiment, the treatment methods comprise determining the expression of at least one splice variant of between at least two and about 1000, more preferably between at least two and about 500, more preferably between at least two and about 250, more preferably between at least two and about 150, more preferably between at least two and about 100, more preferably between at least two and about 75, more preferably between at least two and about 50, more preferably between at least two and about 25, more preferably between at least two and about 10 transcription modulators, wherein expression of each of the transcription modulator splice variants is indicative of cancer.

In another preferred embodiment, the expression of a plurality of splice variants of a transcription modulator is determined. In a preferred embodiment, the expression of between at least two and about 10, more preferably between at least two and about 5 splice variants of a transcription modulator is determined, wherein expression of each of the transcription modulator splice variants is indicative of cancer.

In another preferred embodiment, the treatment methods further comprise diagnosing a cancer subtype, which generally comprises determining the expression of a plurality of transcription modulator splice variants, wherein the presence or absence of expression of each splice variant is indicative of a cancer subtype. In a preferred embedment, the methods comprise determining the expression of at least one splice variant of a plurality of transcription modulators, wherein the presence or absence of expression of each splice variant is indicative of a cancer subtype, and further comprise administering to the patient a bioactive agent capable of inhibiting the activity of one or more such splice variants determined to be expressed. In another preferred embodiment, the methods comprise determining the expression of a plurality of splice variants of a transcription modulator, wherein the presence or absence of expression of each splice variant is indicative of a cancer subtype, and further comprise administering to the patient a bioactive agent capable of inhibiting the activity of one or more such splice variants determined to be expressed. In a preferred embodiment, the cancer subtype is characterized by metastatic potential. In another embodiment, the cancer subtype is characterized by its refractory behavior, particularly its tolerance to a therapeutic agent. In another preferred embodiment, the cancer subtype is characterized by its invasive activity. In a preferred embodiment, the methods further comprise determining the expression of additional markers which are not transcription modulator splice variants but which are useful markers of tumor cell subtypes. Examples of such markers include integrins, receptors for extracellular signals including receptor tyrosine kinases, non-receptor tyrosine kinases, matrix metalloproteinases, and other molecules known to have a role in signal transduction, cell proliferation, cell motility, cell adhesion, or cell survival.

### BRIEF DESCRIPTION OF THE DRAWINGS

FIG. 1. Expression of the splice variants of Ash-1 in astrocytomas. RT-PCR was performed using cDNA derived from neural stem cells (NSCs), astrocytes, and astrocytomas (A1, GBM1, GBM2, GBM3, GBM4, GBM5). A comparison of normal and abnormal transcripts is shown on the right. Abbreviations, norm—Ash-1 transcript in normal neural tissue; ND150, ND200, ND250, ND350—Ash-1 splice variants in brain neoplasms; analyses of the lower transcripts revealed appr. 150, 200, 250 and 350 bp deletions, respectively, between the ATG and Stop codons.

FIG. 2. Expression of splice variants of various developmental regulators in lung cancer. RT-PCR was performed using cDNA derived from lung tissue (contr), and lung neoplasms (LC1, LC2, LC3, LC4, LC5). A comparison of normal Irx2a, NeuroD1, NeuroD3, Oct-2, Rest/NRSF/XBR, SMAD-6 transcripts with their respective abnormal transcripts is shown on the right. Abbreviations, norm—Irx2a, NeuroD1, NeuroD3, Oct-2, Rest/NRSF/XBR, SMAD-6 transcripts in normal tissue; ND60, ND150, ND550, ND650—splice variants of NeuroD1, NeuroD3, Oct-2, Irx2a and SMAD-6, respectively in lung neoplasms; analyses of the lower transcripts revealed appr. 60, 150, 550 and 650 bp deletions, respectively, between the ATG and Stop codons; Ni50—Rest/NRSF/XBR splice variant with a 50 bp insertion located between exons 5 and 6.

FIG. 3. Expression of splice variants of various developmental regulators in neuroblastoma. RT-PCR was performed using cDNA derived from neural stem cells (NSCs), adult hippocampal tissue (HC ad), and neuroblastomas (NB1, NB2, NB3, NB4). A comparison of normal Bmp-2, Neu and Rest/NRSF/XBR transcripts with their respective abnormal transcripts is shown on the right. Abbreviations, norm—Bmp-2, Neu and Rest/NRSF/XBR transcripts in normal neural tissue; ND200, ND400—splice variants of Bmp-2 in neuroblastomas; analyses of the lower transcripts revealed appr. 200, and 400 bp deletions between the ATG and Stop codons; NDNHR1—Neu splice variant with a deleted region encoding NHR1 domain; Ni50 —Rest/NRSF/XBR splice variant with a 50 bp insertion located between exons 5 and 6.

FIG. 4 shows the nucleotide sequence of a novel tumor-specific/enriched splice variant of the human neuralized-1 gene. Also shown are primers which may be used to determine the expression of the splice variant. Also shown is the amino acid sequence of the splice variant.

FIG. 5 shows the nucleotide sequence of a novel tumor-specific/enriched splice variant of the human NeuroD1 gene. Also shown are primers which may be used to determine the expression of the splice variant. Also shown is the amino acid sequence of the splice variant.

FIG. 6 shows the nucleotide sequence of a novel tumor-specific/enriched splice variant of the human Irx-2 gene. Also shown are primers which may be used to determine the expression of the splice variant.

FIG. 7 shows the nucleotide sequence of a novel tumor-specific/enriched splice variant of the human Mash-1 gene. Also shown are primers which may be used to determine the expression of the splice variant. Also shown is the amino acid sequence of the splice variant.

### DETAILED DESCRIPTION OF THE PREFERRED EMBODIMENTS

The present disclosure provides methods for diagnosing cancer and cancer subtypes which generally comprise determining the expression of a plurality of tumor-specific/enriched splice variants of transcription modulators. As disclosed and exemplified herein, it is the combined determination of expression of the plurality, or the overall expression pattern, that provides for the very high accuracy of the diagnostic test, and leads to the molecular identification of cancer subtypes.

“Determining the expression” of a transcription modulator splice variant may be done by assaying for the expression of the splice variant in some way, for example, by assaying for the presence of its encoding mRNA, or the presence of translated protein product. Alternatively, expression may be determined indirectly by assaying for indicia of the expression of a splice variant. For example, an assay for an autoantibody that specifically binds to a splice variant but not to a wildtype transcription modulator may be performed, and the results used to infer whether or not the splice variant is expressed.

The term “wildtype” as referring to an isoform of a transcription modulator means an isoform that is expressed in non-tumor cells, though not necessarily exclusively, and is alternatively spliced relative to a tumor-specific or tumor-enriched splice variant isoform of the transcription modulator. The wildtype isoform is often developmentally regulated. Where more than one isoform satisfies these criteria for wildtype, the most prevalent isoform is referred to as the wildtype isoform.

The term “substantially complementary” herein is meant a situation where a probe sequence is sufficiently complementary to the corresponding region of its target sequence and/or another probe to hybridize under the selected reaction conditions. This complementarity need not be perfect; there may be any number of base-pair mismatches that will interfere with hybridization between a probe sequence (e.g., detection region) and its corresponding target sequence or another probe. However, if the degree of non-complementarity is so great that hybridization between a probe and its target cannot occur under even the least stringent of conditions, the probe sequence is considered to be not complementary to the target sequence.

Splice Variants

The prominent product of gene transcription is termed the primary transcript and is a precursor to mRNA. Many primary transcripts contain intervening nucleotide sequences that are not functional in the final mRNA. These intervening, non-functional sequences are called introns, while the sequences of the primary transcript that are preserved in the mature mRNA are called exons. Accordingly, introns are regions of the initial transcript that must be excised during post-transcriptional RNA processing, and exons are regions that are joined together after intron excision. This excision and joining process is called RNA splicing. The actual splicing is performed by a spliceosome, which is a large particulate complex consisting of various proteins and ribonucleoproteins such as snRNAs and snRNPs.

The spliceosome is responsible for cutting the primary transcript at the two exon-intron boundaries called the splice sites. The nucleotide bases of the splice sites on a primary transcript are always the same. The first two nucleotide bases following an exon are always GU, and the last two bases of the intron are always AG. It is important to note that the two sites have different sequences and so they define the ends of the intron directionally. They are named proceeding from left to right along the intron, that is as the 5′(or donor) and the 3′ (or acceptor) sites.

The majority of normal genes are transcribed into a primary transcript that gives rise to a single type of spliced mRNA. In these cases, there is no variation in the splicing of the primary transcript; the same introns for each of the transcripts are spliced out. However, sometimes the primary transcripts of certain genes follow patterns of alternative splicing, where a single gene gives rise to more than one mRNA sequence.

In an embodiment of the invention, “splice variants” relate to the different mRNA sequences that are derived from the same gene as processed by a spliceosome. Accordingly, “splice variants” encompass any situation in which the single primary transcript is spliced in more than one way, and therefore includes splicing patterns where internal exons are substituted, added, or deleted. “Splice variants” also encompass situations where introns are substituted, added or deleted.

It has been discovered that mRNA splicing is changed in a tumor cell compared to a normal cell. Accordingly, the expression of splice variants in a tumor cell is in some way different from that of a normal cell. Changes in the splicing of tumor cells can be brought about by more than one way. For example, tumors can express products that are necessary for splicing (splicing factors, snRNAs and snRNPs) differently than normal cells. Changes in splicing patterns can also be related to mutations in the donor and acceptor sequences of certain genes in a tumor cell, thereby resulting in different splicing start and termination points.

The physiological activity of splice variant products (proteins) and the original product from which they are derived may differ. For example the splice variant could function in an opposite manner or not function at all. In addition, splice variations may result in changes of various properties not directly connected to biological activity of the protein. For example, a splice variant may have altered stability characteristics (half-life), clearance rate, tissue and cellular localization, temporal pattern of expression, up or down regulation mechanisms, and responses to agonists or antagonists.

Transcription Modulator Splice Variants

The term “transcriptional modulator” is to be construed broadly and in a preferred embodiment relates to factors that play a role in regulating gene expression. In some embodiments, a transcriptional modulator can aid in the structural activation of a gene locus. In other embodiments, a transcriptional modulator can assist in the initiation of transcription. In still other embodiments, a transcriptional modulator can process the transcript. The following is a non-exclusive list of possible factors that are considered to be transcriptional modulators.

Transcriptional modulators include factors that alter chromatin structure to permit access of the transcriptional components to the target gene of interest. One group of promoter restructuring factors that perturbs chromatin in an ATP-dependent manner includes NURF, CHRAC, ACF, the SWI/SNF complex, and SWI/SNF-related (RUSH) proteins.

Another group of transcription modulating factors is involved in the recruitment of a TATA-binding protein (TBP)-containing and not-containing (Initiator) complexes. Examples of general initiation factors include: TFIIB, TFIID, TFIIE, TFIIF, and TFIIH. Each of these general initiation factors are thought to function in intimate association with RNA polymerase II and are required for selective binding of polymerase to its promoters. Additional factors such as TATA-binding protein (TBP), TBP-homologs (TRP, TRF2), initiators that coordinate the interaction of these proteins by recognizing the core promoter element TATA-box or initiator sequence and supplying a scaffolding upon which the rest of the transcriptional machinery can assemble are also considered transcription modulating factors.

Further, TBP-associated factors (TAFs) that function as promoter-recognition factors, as coactivators capable of transducing signals from enhancer-bound activators to the basal machinery, and even as enzymatic modifiers of other proteins are also transcriptional modulators. Particular examples of transcriptional modulators include: the TFIIA complex: (TFIIAa; TFIIAb; TFIIAg); the TFIIB complex: (TFIIB; RAP74; RAP30); the TAFIIA complex: (TAFIIAa; TAFIIAb; TAFIIAg); the TAFIIB complex: (TAFIIB; RAP74; RAP30); TAFs forming the TFIID complex (TAFI-15); the TAFIIE complex: (TAFIIEa; TAFIIEb); the TAFIIF complex (p62; p52; MAT1; p34; XPD/ERCC2; p44; XPB/ERCC3; Cdk7; CyclinH); the RNA polymerase II complex: (hRPB1, hRPB2, hRPB3, hRPB4, hRPB5, hRPB6, hRPB7, hRPB8, hRPB9, hRPB10, hRPB11, hRPB12); and others.

Mediators that act as a conserved interface between gene-specific regulatory proteins and the general transcription apparatus of eukaryotes are also considered to be transcriptional modulators. Typically, this type of mediator complex integrates and transduces positive and negative regulatory information from enhancers and operators to promoters. They typically function directly through RNA polymerase II, modulating its activity in promoter-dependent transcription. Examples of such mediators that form coactivator complexes with TRAP, DRIP, ARC, CRSP, Med, SMCC, NAT, include: TRAP240/DRIP250; TRAP230/DRIP240; DRIP205/CRSP200/TRIP2/PBP/RB18A/TRAP220; hRGR1/CRSP150/DRIP150/TRAP170, TRAP150; CRSP130/hSur-2/DRIP130; TIG-1; CRSP1000TRAP100/DRIP100; DRIP97; DRIP92/TRAP95; CRSP85; CRSP77/DRIP77/TRAP80; CRSP70/DRIP70; Ring3; hSRB10/hCDK8; DRIP36/hMEDp34; CRSP34; CRSP33/hMED7; hMED6; hSRB11/hCyclin C; hSOH1; hSRB7; and others. Additional modulators in this class include proteins of the androgen receptor complex, such as: ANPK; ARIP3; PIAS family (PIASa, PIASb, PIASg); ARIP4; and transcriptional co-repressors such as: the N-CoR and SMRT families (NCOR2/SMRT/TRAC1/CTG26/TNRC14/SMRTE); REA; MSin3; HDAC family (HDAC5); and other modulators such as: PC4; MBF1.

Another class of transcriptional modulators comprises enhancer-bound activators and sequence-specific or general repressors. Examples of these modulators include: non-tissue specific bHLHs, such as: USF; AP4; E-proteins (E2A/E12, E47; HEB/ME1; HEB2/ME2/MITF-2A,B,C/SEF-2/TFE/TF4/R8f); TFE family (TFE3, TFEB); the Myc, Max, Mad families; WBSCR14; and others.

Another example of this class of transcriptional modulators is the neuronally enriched bHLHs such as: Neurogenins (Neurogenin-1/MATH4c, Neurogenin-2/MATH4a, Neurogenin-3/MATH4b); NeuroD (NeuroD-1, NeuroD-2, NeuroD-3(6)/my051/NEX1/MATH2/Dlx-3, NeuroD-4/ATH-3/NeuroM); ATHs (ATH-1/MATH1, ATH-5/MATH5); ASHs (ASH-1/MASH1, ASH-2/MASH2, ASCL-3/reserved); NSCLs (NSCL1/HEN1, NSCL2/HEN2), HANDs (Hand1/eHAND/Thing-1, Hand2/dHAND/Thing-2); Mesencephalon-Olfactory Neuronal bHLHs: COE proteins (COE1; COE2/Olf-1/EBF-LIKE3, COE3/Olf-1 Homol/Mmot1); and others.

Other examples of this class of transcriptional modulators includes: the Glia enriched bHLHs, such as: OLIG proteins (Olig1, Olig2/protein kinase C-binding protein RACK17, Olig3), and others; the bHLH family of negative regulators, which include: Ids (Id1, Id2, Id3, Id4), DIP1, HES (HES1, HES2, HES3, HES4, HES5, HES6, HES7, SHARPs (SHARP1/DEC-2/eip1/Stra13, SHARP2/DEC-1/TR00067497_p), Hey/HRT proteins (Hey1/HRT1/HERP-2/HESR-2, Hey2/HRT2/HERP-1, HRT3), and others. There are other bHLHs that fall within this present category of transcriptional modulators, which include: Lyl family (Lyl-1, Lyl-2); RGS family (RGS1, RGSRGS2/GOS8, RGS3/RGP3); capsulin; CENP-B; Mist1; Nhlh1; MOP3; Scleraxis; TCF15; bA305P22.3; lpf-1/Pdx-1/ldx-1/Sff-1/luf-1/Gsf; and others.

Transcription factors belonging to Wnt pathway are also transcriptional modulators of the present class. Examples of such proteins include: b-catenin; GSK3; Groucho proteins (Groucho-1, Groucho-2, Groucho-3, Groucho-4); TCF family (TCF1A, B, C, D, E, F, G/LEF-1; TCF3; TCF4) and others.

Transcription factors belonging to Notch pathway are also transcriptional modulators of the present class. Examples of such proteins include: Delta, Serrate, and Jagged families (DII1, DII3, DII4, Jagged1, Jagged2, Serrate2); Notch family (Notch1, Notch2, Notch3, Notch4, TAN-1); Bearded family (E(spl)ma, E(spl)m2, E(spl)m4, E(spl)m6); Fringe family (Mfng, Rfng, Lfng); Deltex/dx-1; MAML1; RBP-Jk/CBF1/Su(H)/KBF2; RUNX; and others.

Transcription factors belonging to TGFb/BMP pathway are also transcriptional modulators of the present class. Examples of such proteins include: Chordin; Noggin; Follistatin; SMAD proteins (SMAD1, SMAD2, SMAD3, SMAD4, SAMD5, SMAD6, SMAD7, SMAD8, SMAD9, SMAD10); and others.

Transcription factors belonging to Sonic hedgehog pathway are also transcriptional modulators of the present class. Examples of such proteins include: SHH; IHH; Su(fu); GLI family (GLI/GLI1, Gli2, Gli3); Zic family (Zic/Zic1, Zic2, Zic3); and others.

Fork head/winged helix transcription factors are also transcriptional modulators of the present class. Examples of such proteins include: BF-1; BF-2/Freac4; Fkh5/Foxb1/HFH-e5.1/Mf3; Fkh6/Freac7; and others.

HMG transcription factors are also transcriptional modulators of the present class. Examples of such proteins include: Sox proteins (Sox1, Sox2, Sox3, Sox4, Sox6, Sox10, Sox11, Sox13, Sox14 Sox18, Sox21, Sox22, Sox30); HMGIX; HMGIC; HMGIY; HMG-17; and others.

Homeodomain transcription factors pathway are also transcriptional modulators of the present class. Examples of such proteins include: Hox proteins; Evx family (Evx1, Evx2); Mox family (Mox1, Mox2); NKL family (NK1, NK3, Nkx3.1, NK4); Lbx family (Lbx1, Lbx2); Tlx family (Tlx1, Tlx2, Tlx3); Emx/Ems family (Emx1, Emx2); Vax family (Vax1, Vax2); Hmx family (Hmx1, Hmx2, Hmx3); NK6 family (Nkx6.1); Msx/Msh family (Msx-1, Msx-2); Cdx (Cdx1, Cdx2); Xlox family (Lox3); Gsx family (Goosecoid, GSX, GSCL); En family (En-1, En-2) HB9 family (Hb9/HLXB9); Gbx family (Gbx1,Gbx2), Dbx family (Dbx-1, Dbx-2); DII family (Dlx-1, Dlx-2, Dlx-4, Dlx-5, Dlx-7); Iroquois family (Xiro1, Irx2, Irx3, Irx4, Irx5, Irx6); Nkx (Nkx2.1/TTF-1, Nkx2.2/TTF-2, Nkx2.8, Nkx2.9, Nkx5.1, Nkx5.2); PBC family (Pbx1a, Pbx1b, Pbx2, Pbx3); Prd family (Otx-1, Otx-2, Phox2a, Phox2B); Ptx family (Pitx2, Pitx3/Ptx3), XANF family (Hesx1/XANF-1); BarH family (BarH, Brx2); Cut; Gtx; and others.

POU domain factors are also transcriptional modulators of the present class. Examples of such proteins include: Brn2/XIPou2; Brn3a, Brn3b; Brn4/POU3F4; Brn5/Pou6F1; N-Oct-3; Oct-1; Oct-2, Oct2.1, Oct2B; Oct4A, Oct4B; Oct-6; Pit-1; TCFbeta1; vHNF-1A, vHNF-1B, vHNF-1C; and others.

Transcription factors with homeodomain and LIM regions are also transcriptional modulators of the present class. Examples of such proteins include: Isl1; Lhx2; Lhx3; Lhx4; Lhx5; Lhx6; Lhx7 Lhx9; LMO family (LMO1, LMO2, LMO4); and others.

Paired box transcription factors are also transcriptional modulators of the present class. Examples of such proteins include: Pax2; Pax3; Pax5; Pax6; Pax7; Pax8; and others.

Zinc finger transcription factors are also transcriptional modulators of the present class. Examples of such proteins include: GATA family (Gata1, Gata2, Gata3, Gata4/5, Gata6); MyT family (MyT1, MyT1l, MyT2, MyT3); SAL family (HSal1, Sal2, Sal3); REST/N RSF/XBR; Snail family (Scratch/Scrt); Zf289; FLJ22251; MOZ; ZFP-38/RU49; Pzf; Mtsh1/teashirt; MTG8/CBF1A-homolog; TIS11D/BRF2/ERF2; TTF-I interacting peptide 21; Znf-HX; Zhx1; KOX1/NGO-St-66; ZFP-15/ZN-15; ZnF20; ZFP200; ZNF/282; HUB1; Finb/RREB1; Nuclear Receptors (liganded: ER family; TR family; RAR familiy; RXR family; PML-RAR family; PML-RXR family; orphan receptors: Not1/Nurr; ROR; COUP-TF family (COUP-TF1, COUP-TF2)) and others.

RING finger transcription factors are also transcriptional modulators of the present class. Examples of such proteins include: KIAA0708; Bfp/ZNF179; BRAP2; KIAA0675; LUN; NSPc1; Neuralized family (neu/Neur-1, Neur-2, Neur-3, Neur-4); RINGLA; SSA1/RO52; ZNF173; PIAS family (PIAS-a, PIAS-b, PIAS-g, PIAS-g homolog); parkin family; ZNF127 family and others.

Another class of transcriptional modulators includes proteins that are involved in recombination and repair of damaged DNA and in meiotic recombination. Examples of such proteins include: PCNA; RPA (RPA 14 kD, RPA binding co-activator); RFC(RFC 140 kD, RFC 40 kD, RFC 38 kD, RFC 37 kD, RFC 36 kD, RFC/activator homologue RAD17); RAD 50 (RAD 50, RAD 50 truncated, RAD 50-2); RAD 51 (RAD 51, RAD 51 B, RAD 51 C, RAD 51 C truncated, RAD 51 D, RAD 51H2, RAD 51H3, RAD 51 interacting/PIR 51, XRCC2, XRCC3); RAD 52 (RAD 52, RAD 52 beta, RAD 52 gamma, RAD 52 delta); RAD 54 (RAD 54, RAD 54 B, RAD 54, ATRX); Ku (Ku p70/p80); NBS1 (nibrin); MRE11 (MRE11, MRE11A, MRE11 B); XRCC4; and others.

Another class of transcriptional modulators includes proteins relating to cell-cycle progression-dedicated components that are part of the RNA polymerase II transcription complex. Examples of these proteins include: E2F family (E2F-1, E2F-3, E2F-4, E2F-5); DP family (DP-1, DP-2); p53 family (p53, p63; p73); mdm2; ATM; RB family (RB, p107, p130).

Still another class of transcriptional modulators includes proteins relating to capping, splicing, and polyadenylation factors that are also a part of the RNA polymerase II modulating activity. Factors involved in splicing include: Hu family (HuA, HuB, HuC, HuD); Musashil; Nova family (Nova1, Nova2); SR proteins (B1C8, B4A11, ASF SRp20, SRp30, SRp40, SRp55, SRp75, SRm160, SRm300); CC1.3/CC1.4; Def-3/RBM6; SIAHBP/PUF60; Sip1; C1QBP/GC1Q-R/HABP1/P32; Staufen; TRIP; Zfr; and others. Polyadenylation factors include: CPSF; Inducible poly(A)-Binding Protein (U33818), and others.

Another class of transcriptional modulators includes protein kinases. Examples of these proteins include: AGC Group: AGC Group I (cyclic nucleotide regulated protein kinase (PKA & PKg) family); AGC Group II (diacylglycerol-activated/phospholipid-dependent protein kinase C (PKC) family); AGC Group III (related to PKA and PKC (RAC/Akt) protein kinase family); AGC Group IV (kinases that phosphorylate ribosomal protein S6 family); AGC Group V (budding yeast AGC-related protein kinase family); AGC Group VI (kinases that phosphorylate ribosomal protein S6 family); AGC Group VII (budding yeast DB 2/20 family); AGC Group VII (flowering plant PVPk1 protein kinase homologue family); AGC Group Other (other AGC related kinase families); CaMK Group: CaMK Group I (kinases regulated by Ca2+/CaM and close relatives family); CaMK Group II (KIN1/SNF1/Nim1 family); CaMK Other (other CaMK related kinase families); CMGC Group: CMGC Group I (cyclin-dependent kinases (CDKs) and close relatives family); CMGC Group II (ERK (MAP) kinase family); CMGC Group III (glycogen synthase kinase 3 (GSK3) family); CMGC Group IV (casein kinase II family); CMGC Group V (Clk family); CMGC Group Other; Protein-tyrosine kinases (PTK): A. non-membrane spanning: PTK group I (Src family); PTK group II (Tec/Akt family); PTK group III (Csk family); PTK group IV Fes (Fps) family; PTK group V (AbI family); PTK group VI (Syk/ZAP70 family); PTK group VII (Ack family); PTK group IX (focal adhesion kinase (Fak) family); B. membrane spanning: PTK group X (epidermal growth factor receptor family); PTK group XI (Eph/Elk/Eck receptor family); PTK group XII (Axl family); PTK group XIII (Tie/Tek family); PTK group XIV (platelet-derived growth factor receptor family); PTK group XV (fibroblast growth factor receptor family); PTK group XVI (insulin receptor family); PTK group XVII (LTK/ALK family); PTK group XVIII (Ros/Sevenless family); PTK group XIX (Trk/Ror family); PTK group XX (DDR/TKT family); PTK group XXI (hepatocyte growth factor receptor family); PTK group XXII (nematode Kin15/16 family); PTK other membrane spanning kinases (other PTK kinase families); OPK Group: OPK Group I (Polo family); OPK Group II (MEK/STE7 family); OPK Group III (PAK/STE20 family); OPK Group IV (MEKK/STE11 family); OPK Group V (NimA family); OPK Group VI (wee1/mik1 family); OPK Group VII (kinases involved in transcriptional control family); OPK Group VII (Raf family); OPK Group IX (Activin/TGFb receptor family); OPK Group X (flowering plant putative receptor kinases and close relatives family); OPK Group XI (PSK/PTK “mixed lineage” leucine zipper domain family); OPK Group XII (casein kinase I family); OPK Group XII (PKN prokaryotic protein kinase family); OPK Other (other protein kinase families).

Another class of transcriptional modulators includes cytokines and growth factors. Examples of these proteins include: Bone morphogenetic proteins: Decapentaplegic protein (Dpp), BMP2, BMP4; 60A, BMP5, BMP6, BMP7/OP1, BMP8a/OP2 BMP8b/OP3; BMP3 (Osteogenin), GDF10; BMP9, BMP10, Dorsalin-1; BMP12/GDF7 BMP13/GDF6; GDF5; GDF3Ngr2; Vg1, Univin; BMP14, BMP15, GDF1, Screw, Nodal, XNrl-3, Radar, Admp; Cytokines: Ciliary neurotrophic factor (CNTF) family; Leukemia inhibitory factor; Cardiotrophin-1; Oncostatin-M; Interleukin-1 family; Interleukin-2 family; Interleukin-3 (IL-3); Interleukin-4 (IL-4); Interleukin-5 (IL-5) family; Interleukin-6 (IL-6) family; Interleukin-7 (IL-7); Interleukin-9 (IL-9); Interleukin-10 (IL-10); Interleukin-11 (IL-11); Interleukin-12 (IL-12); Interleukin-13 (IL-13); Interleukin-15 (IL-15) family; GM-CSF; G-CSF; Leptin; Epidermal growth factors: Amphiregulin; Acetylcholine receptor-inducing activity (ARIA); Heregulin (Neuregulin) (NEU differentiation factor); Transforming growth factor a (TGF-a) family; Neuregulin 2; Neuregulin 3; Netrin 1 and 2; Fibroblast growth factors (FGF): FGF-1 (acidic); FGF 2 (basic); FGF3/int-2 (murine mammary tumor virus integration site (v-int-2) oncogene homolog); FGF4/transforming gene from human stomach-1/hst/hst-1/heparin-binding secretary transforming factor-1 (HSTF1)/Kaposi's sarcome FGF (ksFGF)/K-FGF/KS3; FGF5/oncogene encoding fibroblast growth factor-related protein; FGF6/fibroblast growth factor-related gene/hst-2; FGF7, keratinocyte growth factor (KGF); FGF8/androgen-induced growth factor (AIGF); FGF9/glia-activating factor (GAF); FGF10/keratinocyte growth factor 2, KGF-2; FGF11/fibroblast growth factor homologous factor 3 (FHF-3); FGF12/fibroblast growth factor homologous factor 1 (FHF-1); FGF13/fibroblast growth factor homologous factor 2 (FHF-2); FGF14/fibroblast growth factor homologous factor 4 (FHF-4); FGF15; FGF16; FGF17/FGF13; FGF18; FGF19; FGF20/XFGF-20; FGF21; FGF22; FGF23; FGFH/fibroblast growth factor homologous; C05D/1.4/hypothetical 48.1 KD protein COD11.4; GDNF: Artemin; Glial-derived neurotrophic factor (GDNF); Neurturin; Persephin; Heparin-binding growth factors: Pleiotrophin (NEGF1); Midkine (NEGF2), Insulin-like growth factors (IGF): Insulin-like IGF1 and IGF2; Neurotrophins: Nerve growth factor (NGF); Brain-derived neurotrophic factor (BDNF); Neurotrophin-3 (NT-3); Neurotrophin-4/5 (NT-4/5); Neurotrophin-6 (NT-6) family; Tyrosine kinase receptor ligands: Stem cell factor; Agrin; FLT3L; Macrophage colony stimulating factor-1 (CSF-1); Platelet derived growth factor (PDGF) family; Other: Hedgehog family (Indian hedgehog (Ihh), Desert Hedgehog (Dhh), Sonic Hedgehog (Shh)); Wnt Group: WNT1/INT; WNT2/IRP, WNT2B/13; WNT3; WNT3A; WNT4; WNT5A, WNT5B; WNT6; WNT7A, WNT7B; WNT8A/WNT8d, WNT8B; WNT10A, WNT10B; WNT11; WNT14; WNT15; WNT16 isoforms; negative regulators of Wnt signaling: Dickkopf (Dkk) family (Dkk1, Dkk2, Dkk3, Dkk4); Frisbee; Cerberus; Wnt binding factors: WIFs.

In a preferred embodiment of the invention, the tumor-specific splice variants of the above listed transcriptional modulators can be used to classify tumors at a molecular level. In other preferred embodiments, these splice variants can be used to diagnose, make a prognosis, identify a treatment and treat neoplastic conditions.

Methods and Compositions for Cancer Diagnosis

Disclosed herein are methods and compositions for the diagnosis of cancer. The methods generally comprise determining the expression of a plurality of tumor-specific./enriched splice variants of transcription modulators. In a preferred embodiment, the methods comprise determining the expression of at least one splice variant of a plurality of transcription modulators, wherein the expression of each splice variant is indicative of cancer. In another preferred embodiment, the methods comprise determining the expression of a plurality of splice variants of at least one transcription modulator.

While the expression of each of the splice variants is indicative of cancer, each is not necessarily expressed in every occurrence of a particular cancer or in every cancer type. Moreover, all splice variants for which expression is determined in a diagnostic assay that gives a result indicative of cancer are not necessarily expressed. Rather, it is the determination of the overall expression pattern of a plurality of tumor-specific/enriched splice variants that provides for the very high accuracy of the subject diagnostic methods. Further, as also exemplified herein, the determination of negative expression results for transcription modulator splice variants in some samples in a cancer group yields the molecular identification of cancer subtypes.

Disclosed herein are sets of transcription modulator splice variants that are tumor-enriched or tumor-specific, the expression of which can be determined, and such a determination used as a highly accurate indicator of cancer. While these particular splice variants are of tremendous utility, other tumor-specific/enriched splice variants are contemplated for use in the subject methods. Disclosed herein is an example exemplifying methods for identifying such transcription modulator splice variants that are useful in the subject methods. It will be appreciated by the artisan that by increasing the number of tumor-specific/enriched splice variants for which expression is determined, the accuracy of the subject methods is increased, and, importantly, cancer subtypes are more clearly defined, and new subtypes are revealed. All of these factors are beneficial to the effective treatment of cancer.

Also as exemplified herein, the determination of the expression of an appropriate combination of tumor-specific/enriched splice variants may be used for the diagnosis a variety of cancers. Thus the present disclosure reveals molecular abnormalities common to a variety of cancer types.

In addition, it will be appreciated by the artisan that the number of tumor-specific/enriched splice variants for which expression is determined can easily be increased to the point where a single, simultaneous expression determination, or a series of expression determinations, is sufficient to diagnose any of a large number of cancer types and subtypes.

Accordingly, the disclosed methods are useful for diagnosing the existence of a neoplasm or tumor of any origin. For example, the tumor may be associated with lung cancer (e.g., small cell lung cancer, non-small cell lung cancer), gastrointestinal cancer (e.g., colorectal cancer, stomach cancer, liver cancer, pancreatic cancer, and cancers of other regions of gastrointestinal tract), breast cancer, prostate cancer, skin cancer (e.g., basal cell carcinoma, melanoma), sarcoma, endocrine cancer (e.g., carcinoids, insulinoma, cancer of thyroid gland), neural cancers (e.g., neuroblastoma, glioblastoma, medulloblastoma, retinoblastoma), bladder cancer, cervical cancer, renal cancer, hematopoietic cancers (e.g., lymphoma, leukemia). In addition to diagnosing general types of tumors, it is a preferred embodiment of the current invention to diagnose molecular subtypes of the above-listed neoplasia and tumors.

In a preferred embodiment of diagnosing a tumor a practitioner could use one of the primers provided herein to detect the expression of tumor-specific/enriched transcriptional modulator splice variants. In another preferred embodiment, a practitioner could diagnose cancer from neoplastic cells from one of the following sources: blood, tears, semen, saliva, urine, tissue, serum, stool, sputum, cerebrospinal fluid and supernatant from cell lysate. However, diagnosis of a tumor can be performed with as few as one tumor cell from any sample source.

The determination of splice variant isoform expression and its distinction from wildtype expression may be accomplished in a number of ways. With respect to autoantibody detection, when alternative splicing produces a splice variant with a coding sequence that differs from the wildtype isoform, peptides unique to the splice variant isoform (i.e., not present in wildtype isoform) may be used to probe patient sera for the presence of autoantibodies that specifically recognize the peptide, where the presence of such antibodies is indicative of the presence of the splice variant irrespective of the presence of the wildtype isoform of the transcription modulator.

With respect to mRNA detection, RT-PCR reactions may be designed to distinguish the presence of splice variant mRNA from wildtype mRNA. In one embodiment, where alternative splicing removes nucleotide sequence present in the wildtype transcript, primers complementary to mRNA sequence adjacent to the splice junction site in the splice variant may be used to generate a PCR product that traverses the junction site to produce a first product, where the same primers would produce a second product of a different size when reacted with a wildtype transcript. PCR products may be distinguished, for example, by size, and the expression of splice variant mRNA may be discerned from the presence of the splice variant-derived PCR product. In another embodiment, where alternative splicing adds sequence not present in the wildtype construct, primers complementary to mRNA sequence adjacent to each of two splice junctions in a splice variant (between which non-wildtype sequence resides) may be used to generate a PCR product that traverses the junction sites of the splice variant to produce a first product, where the same primers would produce a second product of a different size when reacted with a wildtype transcript. Again, PCR products may be distinguished and the expression of splice variant mRNA determined. Alternatively, a first primer complementary to mRNA sequence adjacent to one of the splice junctions may be used with a second primer complementary to a segment of the non-wildtype sequence present in the splice variant. In this case, the second primer would not hybridize to the wildtype construct, and the PCR reaction would only produce a product in the presence of the splice variant. In preferred embodiments, the mRNA sequence adjacent to the splice junction(s) of interest may optimally be within about 50 to about 100 nucleotides of the splice junction(s), though it will be appreciated by the skilled artisan that greater and shorter distances from the splice junction(s) may be used, and such distances are embraced by other embodiments.

PCR methods are well known in the art. For example, see Current Protocols in Molecular Biology, Greene Pub. Associates and Wiley-Interscience; New York; Eds. Ausubel et al., 1988/April 2003, Chapter 15, The Polymerase Chain Reaction.

Additionally, with respect to mRNA detection, oligonucleotide probes that hybridize to sequence unique to a splice variant (i.e. not present in a wildtype transcript) may be used to selectively detect expression of a splice variant of a transcription modulator. Such an approach is possible where alternative splicing generates a splice variant that contains a sequence insertion that is not present in the wildtype isoform of the transcription modulator. Such oligonucleotide probes are well suited for use in an array. An array may contain a plurality of such splice-variant specific oligonucleotide probes, and may contain probes for additional factors whose expression determination is of use in cancer diagnosis or prognosis, or provides relevant pharmacogenetic information, for example, how a patient will metabolize a particular drug.

The formation and use of nucleic acid arrays is well known in the art. For example, see Current Protocols in Molecular Biology, Greene Pub. Associates and Wiley-Interscience; New York; Eds. Ausubel et al., 1988/April 2003, Chapter 22, Nucleic Acid Arrays.

Autoantibody Detection Platforms

ELISA methods, and array-based protein detection methods are well known to those skilled in the art. Peptides for the detection of autoantibodies specific for tumor-enriched or tumor-specific transcription modulator splice variants may be non-diffusibly bound to an insoluble support having isolated sample receiving areas (e.g., a microtiter plate, an array, etc.). The insoluble supports may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, Teflon™, etc. Microtiter plates and arrays are especially convenient because a large number of assays can be carried out simultaneously, using small amounts of reagents and samples. In some cases magnetic beads and the like are included. The particular manner of binding of the composition is not crucial so long as it is compatible with the reagents and overall methods of the invention, maintains the activity of the composition and is nondiffusable. Preferred methods of binding include direct binding to “sticky” or ionic supports, chemical crosslinking, the synthesis of peptide on the surface, etc. Following binding of the peptide, excess unbound material is removed by washing. The sample receiving areas may then be blocked through incubation with bovine serum albumin (BSA), casein or other innocuous protein or other moiety.

Methods and Compositions for Cancer Subtype Diagnosis and Prognosis

It is a further embodiment of the present invention that the disclosed methods of diagnosing and classifying tumors be used by a practitioner to make a prognosis of a neoplastic condition. Because the developmental stage of any particular cell type is characterized by the expression of a unique set of active transcriptional modulators, assaying the expression of transcriptional modulator splice variants would allow a practitioner to foretell the course of a particular tumor, and/or monitor the course of an ongoing therapeutic regimen.

Diagnostic and Prognostic Kits

The present invention also encompasses kits for performing the diagnostic and prognostic methods of the invention. Such kits can be prepared from readily available materials and reagents. For example, such kits can comprise any one or more of the following materials: enzymes, reaction tubes, buffers, detergent, primers and probes. It is preferred that these test kits contain one or more of the primer sequences provided herein to be used to detect the presence of tumor-specific/enriched transcriptional modulator splice variants. In a preferred embodiment, these test kits allow a practitioner to obtain samples of neoplastic cells in blood, tears, semen, saliva, urine, tissue, serum, stool, sputum, cerebrospinal fluid and supernatant from cell lysate. In another preferred embodiment these test kits include the needed apparatus for performing RNA extraction, RT-PCR, and gel electrophoresis. Instructions for performing the assays can also be included in the kits.

Therapeutics and Methods of Treatment

Also disclosed herein are methods for the treatment of cancer, and bioactive agents useful in these methods. Bioactive agents are agents having biological activity. Specifically, they are chemical entities that are capable of reacting with one or more molecules in a cell or in an organism to produce an effect in that cell or organism.

Bioactive agents encompass numerous chemical classes, though typically they are organic molecules, preferably small organic compounds having a molecular weight of more than 100 and less than about 2,500 daltons, more preferably between 100 and 2000, more preferably between about 100 and about 1250, more preferably between about 100 and about 1000, more preferably between about 100 and about 750, more preferably between about 200 and about 500 daltons. Bioactive agents comprise functional groups necessary for structural interaction with proteins, particularly hydrogen bonding, and typically include at least an amine, carbonyl, hydroxyl or carboxyl group, preferably at least two of the functional chemical groups. The bioactive agents often comprise cyclical carbon or heterocyclic structures and/or aromatic or polyaromatic structures substituted with one or more of the above functional groups. Bioactive agents are also found among biomolecules including peptides, saccharides, fatty acids, steroids, purines, pyrimidines, derivatives, structural analogs or combinations thereof. Preferred bioactive agents include peptides, e.g., peptidomimetics. Peptidomimetics can be made as described, e.g., in WO 98/56401.

Bioactive agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In a preferred embodiment, the bioactive agents are organic chemical moieties or small molecule chemical compositions, a wide variety of which are available in the literature.

In another preferred embodiment, the bioactive agents are nucleic acids. By “nucleic acid” or oligonucleotide or grammatical equivalents herein means at least two nucleotides covalently linked together. A nucleic acid of the present invention will generally contain phosphodiester bonds, although in some cases, as outlined herein, particularly with respect to antisense nucleic acids or probes, nucleic acid analogs are included that may have alternate backbones, comprising, for example, phosphoramide (Beaucage, et al., Tetrahedron, 49(10):1925 (1993) and references therein; Letsinger, J. Org. Chem., 35:3800 (1970); Sprinzl, et al., Eur. J. Biochem., 81:579 (1977); Letsinger, et al., Nucl. Acids Res., 14:3487 (1986); Sawai, et al., Chem. Lett., 805 (1984), Letsinger, et al., J. Am. Chem. Soc., 110:4470 (1988); and Pauwels, et al., Chemica Scripta, 26:141 (1986)), phosphorothioate (Mag, et al., Nucleic Acids Res., 19:1437 (1991); and U.S. Pat. No. 5,644,048), phosphorodithioate (Briu, et al., J. Am. Chem. Soc., 111:2321 (1989)), O-methylphophoroamidite linkages (see Eckstein, Oligonucleotides and Analogues: A Practical Approach, Oxford University Press), and peptide nucleic acid backbones and linkages (see Egholm, J. Am. Chem. Soc., 114:1895 (1992); Meier, et al., Chem. Int. Ed. Engl., 31:1008 (1992); Nielsen, Nature, 365:566 (1993); Carlsson, et al., Nature, 380:207 (1996), all of which are incorporated by reference)). Other analog nucleic acids include those with positive backbones (Denpcy, et al., Proc. Natl. Acad. Sci. USA, 92:6097 (1995)); non-ionic backbones (U.S. Pat. Nos. 5,386,023; 5,637,684; 5,602,240; 5,216,141; and 4,469,863; Kiedrowshi, et al., Angew. Chem. Intl. Ed. English, 30:423 (1991); Letsinger, et al., J. Am. Chem. Soc., 110:4470 (1988); Letsinger, et al., Nucleoside & Nucleotide, 13:1597 (1994); Chapters 2 and 3, ASC Symposium Series 580, “Carbohydrate Modifications in Antisense Research”, Ed. Y. S. Sanghui and P. Dan Cook; Mesmaeker, et al., Bioorganic & Medicinal Chem. Lett., 4:395 (1994); Jeffs, et al., J. Biomolecular NMR, 34:17 (1994); Tetrahedron Lett., 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars, as well as “locked nucleic acids”, are also included within the definition of nucleic acids (see Jenkins, et al., Chem. Soc. Rev., (1995) pp. 169-176). Several nucleic acid analogs are described in Rawls, C & E News, Jun. 2, 1997, page 35. All of these references are hereby expressly incorporated by reference. These modifications of the ribose-phosphate backbone may be done to facilitate the addition of additional moieties such as labels, or to increase the stability and half-life of such molecules in physiological environments. In addition, mixtures of naturally occurring nucleic acids and analogs can be made. Alternatively, mixtures of different nucleic acid analogs, and mixtures of naturally occurring nucleic acids and analogs may be made. The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc.

Examples of highly preferred bioactive agents are described below, though this description is in no way to be construed as limiting the set of bioactive agents useful in the present methods.

(i) siRNA

Inhibition of the activity of specific isoforms of transcription modulators, particularly tumor-specific or tumor-enriched splice variants of transcription modulators, may be accomplished using short interfering RNA (siRNA). Numerous data show that the activity of specific genes and isoforms can be inhibited using siRNA. For example, see Bai et al., Nucleic Acids Res., 31:7264-70, 2003; Wall et al., Lancet., 362:1401-3, 2003; Zhang et al., Cell, 115:177-86, 2003; Quinn et al., Cancer Res., 63:6221-8, 2003.

(ii) Antisense

Inhibition of the activity of specific isoforms of transcription modulators, particularly tumor-specific or tumor-enriched splice variants of transcription modulators, may be accomplished using antisense oligonucleotides. Numerous data show that the activity of specific genes and isoforms can be inhibited using antisense oligonucleotides. For example, see Manion et al., Cancer Biol Ther., 2:S105-14, 2003; Zhang et al., Proc Natl Acad Sci, 100:11636-41, 2003; Kabos et al., J Biol. Chem., 277:8763-6, 2002.

(iii) Intrabodies

The use of intrabodies is known in the art, for example, see Marasco, Curr. Top. Microbiol. Immunol. 260:247-270, 2001; Wirtz et al., Prot. Sci. 8(11):2245-50 (1999); Ohage et al. J. Mol. Biol. 291(5):1129-34 and Ohage et al. J. Biol. Chem. 291(5): 1119-28 (1999). Intrabodies may be used to modulate the activity of transcription modulator splice variants in situ.

(iv) Decoy Nucleic Acids

Inhibition of the activity of specific isoforms of transcription modulators, particularly tumor-specific or tumor-enriched splice variants of transcription modulators, where the transcription modulators are nucleic acid binding proteins, may be accomplished using “decoy” oligonucleotides that specifically bind to the splice variants and inhibit binding to native targets, including regulatory elements in genomic DNA. Numerous data show that the activity of specific genes and isoforms can be inhibited using decoy oligonucleotides. For example, see Cho et al., Proc Natl Acad Sci, 99:15626-31, 2002; Ahn et al., Biochem Biophys Res Commun., 310:1048-53, 2003; Morishita, Curr Drug Targets, 4:2 p before 599, 2003.

(v) Dominant Negative Isoforms

Inhibition of the activity of specific isoforms of transcription modulators, particularly tumor-specific or tumor-enriched splice variants of transcription modulators, may be accomplished using dominant negative isoforms of the transcription modulators. Because much is known about the structure of transcription modulators and the function of individual domains within transcriptional modulators, the function of splice variants can be predicted, and the suitability of the dominant negative technique for the inhibition of splice variant activity can be gauged. Basically, a dominant negative isoform will be designed to lack at least one molecular activity of a targeted splice variant while maintaining other activities and effectively replacing the splice variant with an isoform that is functionally deficient in at least one respect. For example, where the target splice variant is a transcription factor with an identifiable DNA-binding domain, activation domain, and protein:protein interaction motif, a dominant negative may be engineered to maintain the protein:protein interaction motif, but lack the DNA binding domain. Taking the place of the splice variant, the dominant negative will participate in protein:protein interactions with splice variant partners, but be unable to bind DNA as the splice variant normally would. Such a dominant negative design is reminiscent of the Id family of bHLH transcription factor inhibitors.

(vi) Mimicking Peptides

Inhibition of the activity of specific isoforms of transcription modulators, particularly tumor-specific or tumor-enriched splice variants of transcription modulators, may be accomplished using cell penetrating peptides (CPP) containing “mimicking peptides”. “Mimicking peptides” mimick the interaction domains of transcription factors, i.e., exhibit the function of the interaction domain and may take the place of a splice variant in this respect, and are transported into cells by the CPP. Such CPP-mimicking peptide conjugates have been shown to effectively modulate the activity of transcription factors. For example, see Krosl et al., Nat Med., 9:1428-32, 2003; Arnt et al., J Biol. Chem., 15;277(46):44236-43, 2002; Kanovsky et al., Proc Natl Acad Sci, 98(22):12438-43, 2001.

(vii) Small Molecules

Inhibition of the activity of specific isoforms of transcription modulators, particularly tumor-specific or tumor-enriched splice variants of transcription modulators, may be accomplished using small molecules. A small molecule may interfere with any activity possessed by a transcription modulator splice variant that contributes to its ability to modulate transcription. For example, a small molecule may interfere with the ability of a transcription modulator splice variant to enter the nucleus, or to bind DNA, or to heterodimerize with a DNA-binding partner, or to interact with a corepressor molecule, or to interact with a basal transcription factor. Numerous data show that the activity of specific genes and isoforms can be inhibited using small molecules. For example, see Berg et al., Proc Natl Acad Sci, 99:3830-5, 2002; Bykov et al., Nat Med., 8:282-8, 2002.

(viii) Gene Therapy

Where the expression of splice variant transcription modulators endows a tumor cell with a unique transcriptional activity, particularly a transcription activating activity that is mediated by a responsive element in DNA, such activity may be exploited to selectively express toxic agents in tumor cells. Specifically, a recombinant construct comprising a gene encoding a toxic agent under the control of such a responsive element may be engineered and introduced into cells, where it will be selectively expressed in such tumor cells possessing the unique transcriptional activity. Toxic agents may include toxic proteins, peptides, antisense oligonucleotides, and siRNAs. Toxic proteins and peptides are those that are detrimental to cell survival.

By “inhibiting activity” is meant reducing from the activity level observed in the absence of the bioactive agent, including reducing activity to an undetectable level of activity.

Pharmaceutical Compositions and Treatment

The bioactive agents, either alone or in combination, may be used in vitro, ex vivo, and in vivo depending on the particular application. In accordance, the present invention provides for administering a pharmaceutical composition comprising a pharmaceutically acceptable carrier and a pharmacologically effective amount of one or more of the bioactive agents. The pharmaceutical composition may be formulated as powders, granules, solutions, suspensions, aerosols, solids, pills, tablets, capsules, gels, topical cremes, suppositories, transdermal patches (e.g., via transdermal iontophoresis), etc.

As used herein, “pharmaceutically acceptable carrier” comprises any of standard pharmaceutically accepted carriers known to those of ordinary skill in the art in formulating pharmaceutical compositions. Thus, bioactive agents, by themselves, such as being present as pharmaceutically acceptable salts, or as conjugates, or where appropriate, nucleic acid vehicles encoding bioactive peptides, may be prepared as formulations in pharmaceutically acceptable diluents; for example, saline, phosphate buffer saline (PBS), aqueous ethanol, or solutions of glucose, mannitol, dextran, propylene glycol, oils (e.g., vegetable oils, animal oils, synthetic oils, etc.), microcrystalline cellulose, carboxymethyl cellulose, hydroxylpropyl methyl cellulose, magnesium stearate, calcium phosphate, gelatin, polysorbate 80 or the like, or as solid formulations in appropriate excipients. Other types of suitable carriers include liposomes, microparticles, nanoparticles, hydrogels, as is well known in the art.

The formulations may include bactericidal agents, stabilizers, buffers, emulsifiers, preservatives, sweetening agents, lubricants, or the like. If administration is by oral route, the oligopeptides may be protected from degradation by using a suitable enteric coating, or by other suitable protective means, for example internment in a polymer matrix such as microparticles or pH sensitive hydrogels.

Suitable carriers, including excipients and diluents, may be found in, among others, Remington's Pharmaceutical Sciences, Mack Publishing Co., Philadelphia, Pa. (17th ed., 1985) and Handbook of Pharmceutical Excipients, 3rd Ed, Washington D.C., American Pharmaceutical Association (Kibbe, A. H. ed., 2000); hereby incorporated by reference in their entirety. The pharmaceutical compositions described herein can be made in a manner well known to those skilled in the art (e.g., by means conventional in the art, including, by way of example and not limitation, mixing, dissolving, granulating, levigating, emulsifying, encapsulating, entrapping or lyophilizing processes).

The concentrations of the bioactive agents for use in the methods of treatment described herein will be determined empirically in accordance with conventional procedures for the particular purpose. Generally, for administering the bioactive agents ex vivo or in vivo for therapeutic purposes, the bioactive agents are given at a pharmacologically effective dose. By “pharmacologically effective amount” or “pharmacologically effective dose” is an amount sufficient to produce the desired physiological effect or amount capable of achieving the desired result, particularly for treating the disorder or disease condition, including reducing or eliminating one or more symptoms or manifestations of the disorder or disease.

The effective dose administered to the host will vary depending upon what is being administered, the purpose of the administration, such as prophylaxis or therapy, the state of the host, the manner of administration, the number of administrations, interval between administrations, and the like. These can be determined empirically by those skilled in the art and may be adjusted for the extent of the therapeutic response. Factors to consider in determining an appropriate dose include, but are not limited to, size and weight of the subject, the age and sex of the subject, the severity of the symptom, the stage of the disease, method of delivery of the agent, half-life of the agents, and efficacy of the agents. Stage of the disease to consider includes whether the disease is relapsing or in remission phase, and the progressiveness of the disease. Determining the dosages and times of administration for a therapeutically effective amount are well within the skill of the ordinary person in the art.

For example, an initial effective dose can be estimated initially from cell culture assays. Tumor cell proliferation and/or expression of splice variants of the transcriptional modulators may be used to assay effectiveness of the bioactive agent. A dose can then be formulated in animal models to generate a circulating concentration or tissue concentration, including that of the IC₅₀ (concentration of bioactive reagent to achieve 50% reduction in activity being assayed, e.g., cell proliferation) as determined by the cell culture assays. Useful animal models include, but are not limited to, mouse, rat, guinea pigs, rabbits, pigs, monkeys, and chimpanzees.

In addition, the toxicity and therapeutic efficacy may be determined by cell culture assays and/or experimental animals, typically by determining a LD₅₀ (lethal dose to 50% of the test population) and ED₅₀ (therapeutically effectiveness in 50% of the test population). The dose ratio of toxicity and therapeutic effectiveness is the therapeutic index. Preferred are bioactive agents, individually or in combination, exhibiting high therapeutic indices.

For the purposes of this invention, the methods for administering the bioactive agents are chosen depending on the condition being treated, the form of the bioactive agent, and the pharmaceutical composition. Administration of the bioactive agents can be done in a variety of ways, including, but not limited to, cutaneously, subcutaneously, intravenously, orally, topically, transdermally, intraperitoneally, intramuscularly, and intravesically. For example, microparticle, microsphere, and microencapsulate formulations are useful for oral, intramuscular, or subcutaneous administrations. Liposomes and nanoparticles are additionally suitable for intravenous administrations. Administration of the pharmaceutical compositions may be through a single route or concurrently by several routes. For instance, oral administration can be accompanied by intravenous or parenteral injections.

In one embodiment, the method of administration is by oral delivery, in the form of a powder, tablet, pill, or capsule. Pharmaceutical formulations for oral administration may be made by combining one or more of the bioactive agents with suitable excipients, such as sugars (e.g., lactose, sucrose, mannitol, or sorbitol), cellulose (e.g., starch, methyl cellulose, hydroxymethyl cellulose, carboxymethyl cellulose, etc.), gelatin, glycine, saccharin, magnesium carbonate, calcium carbonate, polymers such as polyethylene glycol or polyvinylpyrrolidone, and the like. The pills, tablets, or capsules may have an enteric coating, which remains intact in the stomach but dissolves in the intestine. Various enteric coating are known in the art, a number of which are commercially available, including, but not limited to, methacrylic acid-methacrylic acid ester copolymers, polymer cellulose ether, cellulose acetate phathalate, polyvinyl acetate phthalate, hydroxypropyl methyl cellulose phthalate, and the like. In another embodiment, oral formulations of the bioactive agents are in prepared in a suitable diluent. Suitable diluents include various liquid forms (e.g., syrups, slurries, suspensions, etc.) in aqueous diluents such as water, saline, phosphate buffered saline, aqueous ethanol, solutions of sugars (e.g., sucrose, mannitol, or sorbitol), glycerol, aqueous suspensions of gelatin, methyl cellulose, hydroxylmethyl cellulose, cyclodextrins, and the like. In some embodiments, lipohilic solvents are used, including oils, for instance, vegetable oils, peanut oil, sesame oil, olive oil, corn oil, safflower oil, soybean oil, etc.; fatty acid esters, such as oleates, triglycerides, etc.; cholesterol derivatives, including cholesterol oleate, cholesterol linoleate, cholesterol myristilate, etc.; liposomes; and the like.

In yet another embodiment, the administration is carried out cutaneously, subcutaneously, intraperitonealy, intramuscularly and/or intravenously. Bioactive agents may be dissolved or suspended in a suitable aqueous medium for administration. Additionally, the pharmaceutical compositions for injection may be prepared in lipophilic solvents, which include, but are not limited to, oils, such as vegetable oils, olive oil, peanut oil, palm oil soybean oil, safflower oil, etc; synthetic fatty acid esters, such as ethyl oleate or triglycerides; cholesterol derivatives, including cholesterol oleate, cholesterol linoleate, cholesterol myristilate, etc.; or liposomes, as described above. The bioactive agents may be prepared directly in the lipophilic solvent or as oil/water emulsions, (see for example, Liu, F. et al., Pharm. Res. 12: 1060-1064 (1995); Prankerd, R. J., J. Parent. Sci. Tech. 44: 139-49 (1990); and U.S. Pat. No. 5,651,991).

The delivery systems also include sustained release or long-term delivery methods, which are well known to those skilled in the art. By “sustained release or” “long term release” as used herein is meant that the delivery system administers a pharmaceutically therapeutic amount of bioactive agent for more than a day, preferably more than a week, and in certain instances 30 days to 60 days, or longer. Long term release systems may comprise implantable solids or gels, such as biodegradable polymers (see, e.g., Brown, D. M. et al., Anticancer Drugs, 7:507-513 (1996)); pumps, including peristaltic pumps and fluorocarbon propellant pumps; osmotic and mini-osmotic pumps; and the like.

Development of a Database

Also contemplated herein is the formation of a database correlating transcription modulator splice variant expression with cancer phenotype and response to treatment. The establishment of such a database provides for the optimization of cancer treatment, whereby a precise molecular cancer diagnosis/prognosis is made by transcription modulator splice variant profiling, and consultation of the database reveals what treatments are likely to benefit the patient, and what treatments are likely to have harmful side effects and/or be ineffective for the patient.

### EXPERIMENTAL

1. Identification of Tumor-Specific/Enriched Splice Variants of Transcription Modulators Useful for Diagnosis

A number of public databases holding gene expression data derived from a variety of cancer types are well known. For example, National Center for Biotechnology Information's EST database houses records of expressed sequence tags (ESTs) identified in differential display experiments, including ESTs that are upregulated or specific to a variety of cancer types.

Based on the identification of such EST sequences, a genomic database (such as that at NCBI) was consulted to identify corresponding genes. Those which were determined by inspection, using knowledge held in the art, to be multi-exon genes encoding transcription modulators, and thus having the potential to generate transcription modulator splice variants specific to or enriched in cancer, were identified. Primers directed to the distal 5′ (at start) and distal 3′ (at stop) regions of mRNA based on the wildtype sequence were used in RT-PCR reactions with RNA isolated from a variety of tumor cell types, including primary human tumor cell samples and human tumor cell lines. PCR products differing from the wildtype-derived product were sequenced and determined to be transcription modulator splice variants expressed in tumor cells.

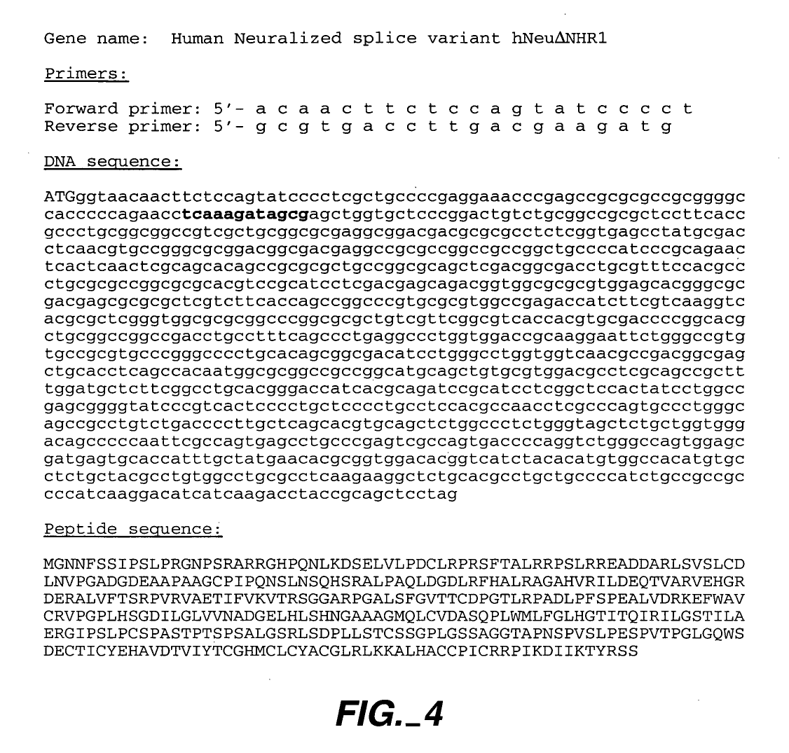

Using this approach, novel tumor-specific/enriched splice variants of the human genes neuralized-1, Irx-2, Mash-1, and NeuroD1 were identified. The nucleotide sequences of these novel splice variant nucleic acids are set forth in FIGS. 4-7. The nucleotide sequences of primers useful for the determination of the expression of these splice variants are also shown in the figures. The amino acid sequences of the splice variants is also shown.

In addition, the following peptides may be used to determine the expression of autoantibodies that specifically bind to these novel splice variants. The peptides bind to the novel splice variants (individually), but do not bind to wildtype isoforms of the corresponding transcription modulators. The peptide GHPQNLKDSELV binds specifically to the neuralized splice variant; the peptide MNAEEBSLRNGG binds specifically to the NeuroD1 splice variant. The peptide MRCKRRLNSSGF binds specifically to the Mash-1 splice variant. The peptide CKRLLFRRMYDR binds specifically to Irx2a splice variant.

In a preferred embodiment, disclosed herein are peptides useful for the detection of the novel splice variants disclosed in FIGS. 4-7. In another preferred embodiment, disclosed herein are peptide arrays comprising the peptides listed above. In another preferred embodiment, disclosed herein are peptide arrays comprising a plurality of peptides, which themselves comprise the peptides listed above. In another preferred embodiment, disclosed herein are peptide arrays comprising a plurality of peptides, which themselves consist essentially of the peptides listed above.

It will be appreciated by the artisan that independent experiments may be done to identify genes that are differentially expressed, and particularly tumor-enriched/specific. Methods for the identification of differentially expressed genes are well known. For example, see Current Protocols in Molecular Biology, Greene Pub. Associates and Wiley-Interscience; New York; Eds. Ausubel et al., 1988/April 2003, Chapter 25, Discovery of Differentially Expressed Genes. This approach is also embraced by the current disclosure.

2. Molecular Classification of Specific Tumors

The examples below demonstrate the molecular classification of specific types of tumors or neoplasms. Specific classes of tumors are subdivided into subclasses based upon the expression patterns of transcriptional modulator splice variants. These subclasses can be used for diagnostic and prognostic purposes. Additionally, the tumor types classified below can be used to identify treatments for and treat neoplastic conditions.

The list of splice variants used in the following examples is not finite. Using the methods disclosed herein, additional splice variants could be added to the arrays illustrated below to expand the classification system and to increase its specificity. Notwithstanding the expandability of the methods disclosed herein, the addition of new transcriptional modulator splice variants to the system does not alter the basic principle of the disclosed invention.

### Example 1

### Expression of Transcriptional Modulator Splice Variants in Glioblastoma Cells

Biopsy samples of glioblastoma cells were obtained from various sources and the RNA was extracted. RT-PCR was used to amplify the corresponding DNA sequences in order to identify transcriptional modulator splice variants.

First strand cDNAs were synthesized with reverse transcriptase (SuperscriptII, Life Technologies Inc.) using 5-10 mg of mRNA from different cell lines as a template. PCR reactions were performed in the volume of 25 ml containing 1/10 of RT reaction as a template and GC-Rich PCR System or the Expand™ Long Distance PCR System kit (Roche) according to manufacturer's instructions. In most cases, the DNA was amplified using the following conditions: 94° C. (2 min); 35-40 cycles of 94° C. (30s), 56° C. (40s), 72° C. (150s). For all combinations of primers the annealing temperature and the number of cycles was optimized beforehand. All amplified PCR products were sequenced to rule out false positives using fmol® DNA Cycle Sequencing System (Promega). The amplified RT-PCR products were then resolved on 1.0-1.2% agarose gel.

These analyses revealed that following genes express tumor specific splice variants in glioblastoma cells: ASH1, NeuroD1, NeuroD3, Oct2, NRSF/REST/XBR, Neuralized 1, and RAD51 B.

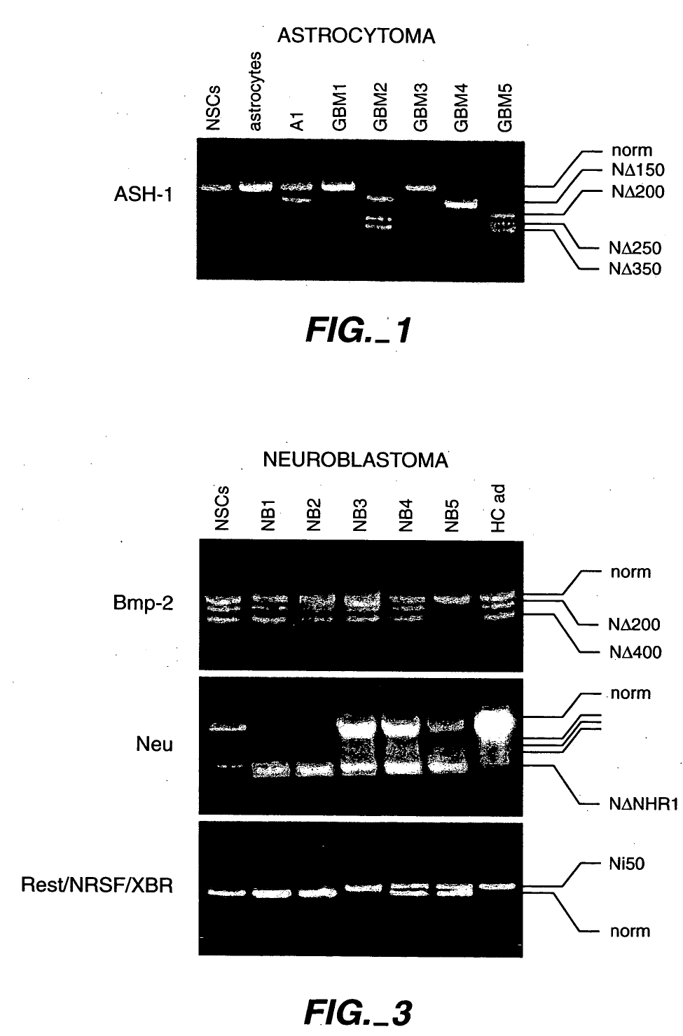

Based on the expression of splice variants it was possible to discriminate between normal and tumor cells and also between different grade astrocytomas such as Glioblastoma multiforma (GBM), anaplastic astrocytoma and grade 2 astrocytoma. For example normal astrocytes express a single splice variant of Helix-Loop-Helix transcription factor ASH1 whereas majority of GBMs express 1-3 splice variants and anaplastic astrocytoma expresses normal and one variant form of ASH1 mRNA (FIG. 1). Based on the expression of transcriptional modulator splice variants, this analysis showed that the samples included at least 3 molecular subtypes of GBMs. Subtype A was characterized as having normal ASH1, normal and NΔ150 NeuroD3 and NΔNHR1 Neu expression, Subtype B was characterized as having NΔ200 ASH1, normal and NΔ150 Oct2 and NΔ60 NeuroD1 expression, and finally Subtype C was characterized as having NΔ150, NΔ250, NΔ350 ASH1, normal Irx2a, Ni50 Rest/NRSF/XBR expression.

All of the RNA was isolated as described in Timmusk et al., Neuron, 10:475489 (1993). RT-PCR analyses was performed as in Palm et al., J. Neurosci., 8:1280-1296 (1998). (Both of which are hereby incorporated by reference in their entirety). The following primers were used to analyze transcriptional modulator splice variants.

TABLE 1
Primer Sequences

| | GenBank | | |
|---|---|---|---|
| Gene Name | ID | Oligo Name | 5′-3′ |
| Ash-1 | U77616 | s 24 | ccctctctgttcctgcacccaagt |
| | | as 26 Stop | ccagttggtgaagtcgagaagctcct |
| BMP-2 | M22489 | s 24 | cggtccttgcgccaggtcctttga |
| | | as 26 | gtactagcgacacccacaaccctcca |
| Irx-2a | U90304 | N s 26 ATG | accggtcgttccgATGgcagtggaga |
| | | as 23 Stop | cgcgTTAaatgtcggacatacct |
| Neuralized | U87864 | s 24 ATG | ccaccatgggtaacaacttctccagt |
| | | as 25 Stop | gctaggagctgcggtaggtcttgat |
| NeuroD1 | D82347 | s 25 | gccccagggttatgagactatcact |
| | | as E 25 Stop | ggtgaaactggcgtgcctCTAatca |
| NeuroD3 | D81215 | s 26 5′UTR | gactccaggagacgatgcgacactca |
| | U69205 | as 26 Stop | gacaggggaggtgaatgaccactgtt |
| Oct-2 | XB1030 | s 25 | cagtgatctggaggagctggagcaa |
| | | as 27 | ggcgatcagcaggatctcctctgaggt |
| RAD51B | U84138 | s E 29 | ccagcatgggtagcaagaaactaaaacga |
| | | as E 25 | ctgtctctaggaatttccataggct |
| RAD52 | NM_{—} | s E 26 | gcaagatgtctgggactgaggaagca |
| | 002879 | as E 26 | gtggcctgagcctcagtaagatggat |
| RFC140 kD | U19720 | s E 28 | ccacgatggtgccctccagcccagcggt |
| | | as E 24 | gcccgagagtcactggttcacatt |
| REST/ | U22314 | s 25 II Fin | gtgaccgctgcggctacaatactaa |
| NRSF/ | U13879 | as 25 VIII | ggacaagtaggatgcttagatttga |
| XBR | U22680 | Fin | |
| SMAD-6 | AF035528 | s 29 ATG | cgtATGttcaggtccaaacgctcggggct |
| | | as 26 Stop | ccgccaCTAtctggggttgttgagga |

### Example 2

### Expression of Transcriptional Modulator Splice Variants in Non-Small Cell Lung Cancer (NSCLC) Cells

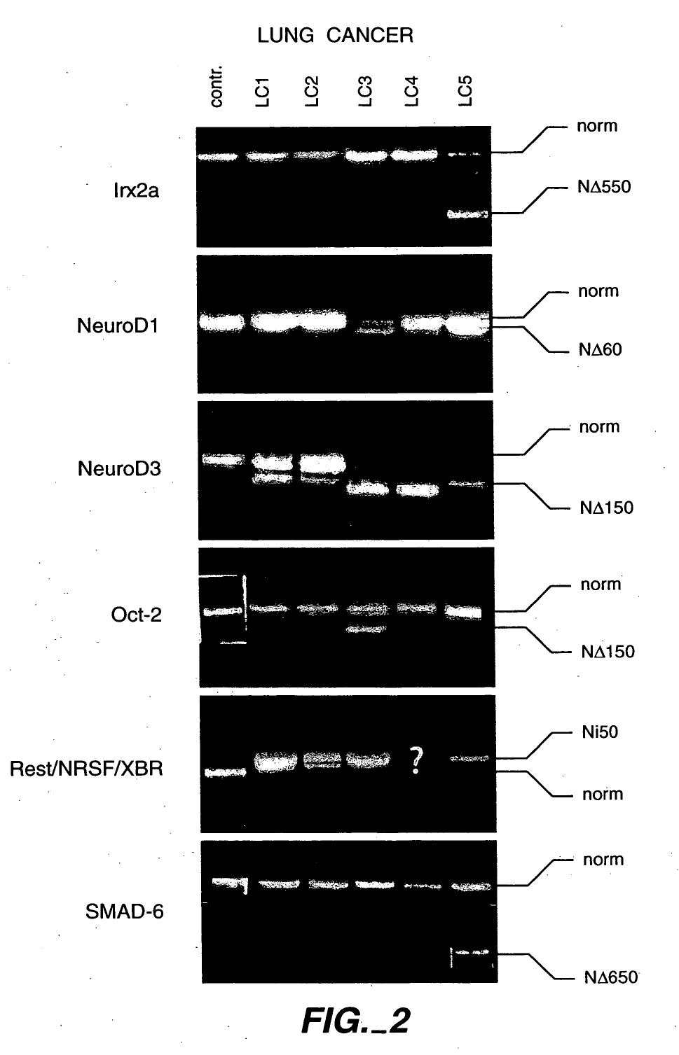

Cell lines and biopsy material of NSCLC cells were used to determine the expression of splice variants. Analyses of NSCLC was performed as described in Example 1. First, RNA was extracted from various tumor sources of the same cell type. RT-PCR was then used to amplify the corresponding DNA, which was subsequently run through gel electrophoresis. The results of this assay are presented in (FIG. 2). The following genes expressed tumor specific splice variants of transcriptional modulators: NeuroD1, NeuroD3, Irx2, NRSF/REST/XBR, Neuralized 1, Oct2, and SMAD-6.

All NSCLC samples express tumor-specific splice variants of NeuroD1 and NRSF/REST/XBR genes. Accordingly, these two markers can be used to identify NSCLC cells in biological specimens. Based on the expression of transcriptional modulator splice variants, this analysis showed that the samples included at least 3 molecular subgroups of NSCLC tumors. Subtype A was characterized as having normal Irx2a, NeuroD3, Oct2 and Smad 6 expression, Subtype B was characterized as having normal Irx2a and Smad 6, NΔ150 Oct2 and NΔ150 NeuroD3 expression, Subtype C was characterized as having normal Irx2a, Oct2, Smad 6 and NΔ150 NeuroD3 expression, and Subtype D was characterized as having Normal Oct2, normal and NΔ550 Irx2a, NΔ150 NeuroD3, normal and NΔ650 Smad6 expression.

### Example 3

### Expression of Transcriptional Modulator Splice Variants in Neuroblastoma Cells

Analyses of neuroblastomas was performed as in Examples 1 and 2. RNA was extracted from various tumor cells of the same cell type. RT-PCR was then used to amplify the corresponding DNA, which was subsequently run through gel electrophoresis. The results are presented in FIG. 3. The following genes expressed tumor-specific transcriptional modulator splice variants: NeuroD1, Ptx3, NRSF/REST/XBR, Neuralized 1, RAD52, and RFC140. All neuroblastomas expressed tumor-specific Neuralized and NRSF/REST/XBR splice variants (FIG. 3) that can be used to detect neuroblastoma cells from biological samples.

### Example 4

### Expression of Transcriptional Modulator Splice Variants in Multiple Tumor Types

As disclosed above, a number of tumor-specific/enriched splice variants from the set of transcription modulators Ash-1, BMP-2, Irx-2a, Neuralized, NeuroD1, NeuroD3, Oct-2, RAD51B, RAD52, RFC140 kD, REST, and SMAD-6 are expressed in neuroblastoma and glioma and non-small cell lung carcinoma. In addition, a number of these splice variants have been observed in prostate cancer cells and breast cancer cells. This data suggests that changes in transcription modulator expression patterns, in part, may be common to a number of different cancers. The establishment of relationships between cancer types gleaned from the profiling of tumor-specific/enriched transcription modulator splice variants as disclosed herein may factor into the design of therapeutics and may be used to optimize treatments.

Preparation of Samples

Blood, ocular discharge, nasal discharge, saliva, feces, CSF, and tissue are collected from healthy and suspected subjects. Peripheral blood mononuclear cells (PBMC) are isolated from 2 ml of whole blood treated with anticoagulant (for example, CPD-A1®, Green Cross Co, Korea) by centrifugation over Ficoll-sodium diatrizoate solution.

Ocular and nasal discharges, saliva, and feces are eluted with 0.5 ml phosphated buffered saline (PBS).

Sputum samples are considered unsatisfactory for evaluation if alveolar lung macrophages are absent or if a marked inflammatory component is present that dilutes the concentration of pulmonary epithelial cells.

Urine often contains very low numbers of tumor cells. In these cases, we recommend concentrating samples of up to 3.5 ml to a final volume of 140 μl, before processing. Concentrated sample of urine are obtained by centrifugation for 10 min at 12,000 rpm. In another application, 30 ml-100 ml of urine samples are spun at 10,000 g, 4° C., 30 min.

Cerebrospinal fluid (CSF) is collected in 0.5 ml samples and processed as non-centrifuged material.

The tumor tissue is obtained through biopsy or surgical resection. For example, tissue samples obtained at resection and biopsies are fixed by perfusion or immersion in neutral buffered formalin (NBF), respectively. A portion of each tumor sample is frozen in liquid nitrogen and the remaining tumor tissue is fixed in NBF, embedded in paraffin; 5-μm sections are cut, and stained with hematoxylin and eosin to identify precursor lesions. Lung lobes obtained from patients undergoing resection were sampled as follows. The normal tissue surrounding the tumor is sampled extending in all directions toward the periphery of the tumor. Approximately eight separate pieces of tissue are embedded in paraffin, sectioned, and stained with hematoxylin and eosin to identify precursor lesions. Lesions are classified based on World Health Organization criteria. Sequential sections from biopsies and lesions identified in resections are cut (5-10 μm), deparaffinized, and stained with toluidine blue to facilitate dissection. A 25-gauge needle attached to a tuberculin syringe is used to remove the lesions under a dissecting microscope. Because of the extensive contamination of some lesions with normal tissue (e.g., SCC, adenoma, alveolar hyperplasia) or the small size of some lesions, <0.001 mm³, it is essential to include normal appearing cells to ensure that enough sample remained to conduct the RT-PCR assay as described below. Since, because the goal of the diagnostic analysis is to determine whether abnormal splice variants are present in these lesions and not to quantitate their levels, the presence of normal tissue-“contaminant” is acceptable. In cases where the lesion is pure, of substantial size (>500 cells), and easily dissected, it is possible to microdissect only the lesion itself.

Laser Capture Microdissection and Immuno-Phenotyping

Laser Capture Microdissection (LCM) and immuno-phenotyping of specific cell types are applied for molecular analyses of a single cell from a heteregoneous mixture of tumor cells from the biopsied material. LCM protocol in brief:

1. Dissociate biopsied material with 0.25% trypsin. Attach dispersed cells to uncoated glass slides by cytocentrifugation. Fix in 100% ethanol 5 minutes. Air dry 5 minutes.

2. Immunostain for general tumor antigens, for example, CEA, PSA, or using an antibody to a common NE marker (chromograninA, synaptophysin, 5-hydroxytryptophan receptor, somatostatin receptor or other). (Negative controls for immunostaining consist of substituting normal serum for the primary antibody, which should result in no staining of the slides.) Lightly counterstain cells with hematoxylin. Place in 3% glycerol in RNase-free water 20 minutes.

3. Dehydrate with 95% and 100% ethanol. Incubate in xylene 10 minutes.

4. Air dry at room temperature. Perform LCM, using 60 mW of laser power and a 30 mm diameter laser beam, for example.

Basic Immunofluorescence Staining and Flow Cytometric Analysis

Basic immunofluorescence staining and flow cytometric analysis protocol can be used for the analysis of surface molecules at single-cell level. Optimal concentration of the fluorochrome-conjugated primary antibodies has to be determined experimentally. To confirm specificity of the staining, it is common to block the directly-conjugated primary antibodies with excess amounts of unlabeled antibody. Alternatively, recombinant peptides can be used for blocking.

Preparation of Target Cells of Interest

Harvest tumor tissue and tease it by pressing with plunger of a syringe or by mashing between two frosted microscope slides using 10 ml of staining buffer (PBS, fetal bovine serum, and sodium azide).

Transfer into a 50 ml conical tube and allow the big clumps and debris to settle to the bottom or run the suspension through a nylon mesh (Falcon cat.no. 2350) to get single cell suspension.

Centrifuge cell suspension 4-5 min (300-400×g) at 4° C., and discard supernatant.

Resuspend the samples in 50 ml of staining buffer and perform a cell count.

Spin cells again, discard supernatant, and resuspend cells in staining buffer at 2×10⁷/ml.

Stain cell-surface antigen following the surface staining protocol. The choice of the surface marker depends on the tumor sample.

Dilute previously determined concentration of primary antibody in 50 μl of staining buffer and dispense to each test tube or well of a microtiter plate. Dispense 50 μl of staining buffer into the unstained or negative control tube.

Add 50 μl of cell suspension (equal to 10⁶ cells) to each tube or well, mix gently.

Incubate at least 20 minutes in the dark on an ice bath or in a refrigerator. The exact conditions of incubation with antibodies are determined in preliminary experiments.

After the incubation period, add staining buffer (2 ml for tubes or 200 μl for microtiter plates).

Centrifuge cells for 5 minutes (300-400×g) at 4° C. Aspirate supernatant.

Repeat 2 times for a total of 3 washes.

If using directly fluorochrome-conjugated antibodies, resuspend stained cell pellet in 500 μl of staining buffer and run on a flow cytometer.

If using purified or biotin conjugated antibodies, add the proper second step (a fluorochrome-conjugated secondary antibody or -Avidin) in 50-100 μl of staining buffer to each sample. Incubate in the dark for 15-30 minutes on an ice bath or in a refrigerator. Wash 2 times as above. Resuspend stained cell pellet in 500 μl of staining buffer and run on a flow cytometer.

For discrimination of viable and dead cells, stain with a viability dye.

Immunocapture

Add 100 μl capture antibody diluted in coating solution to appropriate wells. Antigen or antibody are diluted in coating solution to immobilize them to the microplate. Commonly used coating solutions are: 50 mM sodium carbonate, pH 9.6; 20 mM Tris-HCl, pH 8.5; or 10 mM PBS, pH 7.2. A protein concentration of 1-10 μg/ml is usually sufficient.

Incubate 1 hour at room temperature.

Empty plate and tap out residual liquid.

Add 300 μl blocking solution to each well. Commonly used blocking agents are: BSA, nonfat dry milk, casein, gelatin, etc. Different assay systems may require different blocking agents.

Incubate 5 minutes, empty plate and tap out residual liquid.

Add 100 μl diluted antigen to each well. Primary antibody should be diluted in 1×blocking solution to help prevent non-specific binding. A concentration of 0.1-1.0 μg/ml is usually sufficient.

Incubate at room temperature for 1 hour to overnight.

Empty plate, tap out residual liquid.

Fill each well with wash solution. Typically 0.1 M Phosphate-buffered saline or Tris-buffered saline (pH 7.4) with a detergent such as Tween 20 (0.02%-0.05% v/v).

Invert plate to empty, tap out residual liquid.

Repeat 3 to 5 times.

Captured cells are immediately subjected to RNA extraction.

RNA Extraction

RNA extraction. In a preferred embodiment RNA is extracted from the test and control samples as described in Timmusk et al., Neuron, 10: 475-489 (1993). In brief: To isolate RNA from solid or liquid matrices including blood, stool, sputum, urine, samples are homogenized in 5 ml of Guanidinium lysis buffer (4M Guanidinium isothiocyanate, 25 mM sodium acetate pH 6.0 and 1 mM EDTA pH 8.0; 0.1% DEPC-H₂O; 20% (w/v) N-lauryl sarcosine 10 M; β-mercaptoethanol; 100 mM DTT; RNasin RNase inhibitor (Promega) per 100 μl of the liquid sample, for example. RNA is solubilized by repetitive pipetting. Cell lysates are transferred to a fresh tube and an equal portion (500 μl of the water-saturated acid phenol-chloroform per 100 μl of the liquid sample) is added to the cell lysate. Total RNA is extracted by further ethanol precipitation. In certain applications, liquid matrices (saliva) are first heat-treated (60° C., 15 min) prior to further processing. This is aimed to denature enzymes (salivary) that may affect mRNA stability or interfere with the PCR procedure.

Gene-Specific RT-PCR

cDNA amplification using RT-PCR is performed as is described in Palm et al., J. Neurosci., 8: 1280-1296 (1998). As with any PCR reaction, triplicate samples are run to ensure the validity of the PCR result. Components and cycling will depend on individual template and primers.

1. To RNA pellet, add 10 μl DEPC-H₂O and 1 μl RNase inhibitor (20 U/μl (Perkin Elmer)).

2. Resuspend the RNA pellet with gentle tapping.

3. Quick spin.

4. Aliquot 5 μl into 2 sterile tubes for (+) and (−) RT reactions.

5. For each batch of samples, prepare additional control tubes as follows, using either high-quality RNA or DEPC-dH₂O in place of the 5 μl sample RNA:

| Control Type | (+) RT | (−) RT |
|---|---|---|
| Positive | High-quality RNA | High-quality RNA |
| Negative | DEPC-dH₂O | DEPC-dH₂O |

6. Prepare sufficient volume of the following +/−RT master reaction mixtures for all reaction tubes:

| (+) RT master reaction mixture | | (−) RT master reaction mixture | |
|---|---|---|---|
| 1.0 μl | DEPC-dH₂O | 1.5 μl | DEPC-dH₂O |
| 2.0 μl | First strand RT buffer | 2.0 μl | First strand RT |
| | (Life Technologies) | | buffer (LT) |
| 1.0 μl | dNTP 250 μM (Roche) | 1.0 μl | dNTP 250 μM |
| | | | (Roche) |
| 0.5 μ | Random hexamer primers | 0.5 μl | Random hexamer |
| Total | | Total | primers |
| volume = | | volume = | |
| 4.5 μl | | 5.0 μl | |

7. Aliquot either 4.5 μl or 5.0 μl of the relevant master mix to the (+) and (−) RT tubes.

8. Incubate at 65° C. for 5 minutes, then at 25° C. for 10 minutes.

9. Add 0.5 μl Superscript II (SSII) reverse transcriptase (Life Technologies to all (+) RT tubes only.

10. Incubate all tubes at 25° C. for 10 minutes, then at 37° C. for 40 minutes.

11. Incubate at 95° C. for 5 minutes to denature the SSII.

12. Quick spin.

13. Aliquot 3 μl of each cDNA sample into a sterile PCR tube.

14. Prepare sufficient volume of PCR master reaction mixture for all reaction tubes and add 7 μl to each tube.

PCR Master Reaction Mixture

1.0 μl PCR Buffer GC-Rich PCR System or the Expand™ Long Distance PCR System kit (Roche)

0.8 μl dNTP 250 μM (Roche)

0.2 μl Forward primer

0.2 μl Reverse primer

(0.2 μl dCTP a-³³P (or α-³²P), in cases when necessary)

0.2 μl polymerase, n U/μl, GC-Rich PCR System or the Expand™ Long Distance PCR System kit (Roche), according to manufacturer's instructions

4.6 (4.4) μl DEPC-dH₂O

Total volume=7 μl

15. PCR Cycling Conditions:

The preferred PCR cycling conditions in general are 35 cycles at 920, annealing for 1 minute at 56°, and synthesis for one minute at 72°. A specific example follows.

| Cycles | Temp. (° C.) | Time |
|---|---|---|
| 1 | 94 | 2 min |
| 35-45 | 94 | 30 seconds |
| | X* | 40 seconds |
| | 68 or 72 | 150 seconds |
| 1 | 68 or 72 | 10 min |

56 is annealing temperature, dependent on the primer used.

16. Store the PCR products at 4° C. or continue to step 5.

17. Pour a 1-2% agarose 6% polyacrylamide sequencing gel (PAGE) while the PCR is cycling.

18. After cycling is complete, add 2.5 μl sample buffer (5×) to samples

19. Denature samples at 95° C. for 3 minutes and place directly on ice.

20. Load 3.5 μl sample on gel and run samples to desired distance.

21. Visualize products on an ethidium bromide treated agarose gel or if PAGE is used, then dry gel and expose to phosphoroimager screen or film.

If necessary, RNA from isolated cell populations is then further characterized for purity by reverse transcriptase-polymerase chain reaction (RT-PCR) with primers specific for a series of established marker genes including: vimentin (stromal cells), cytokeratin 19 (glandular epithelial cells) and CD45 (inflammatory cells/lymphocytes), and other. In addition, more specific markers for NE origin of cells (chromograninA, synaptophysin, 5-hydroxytryptophan receptor, somatostatin receptor or other) can be incorporated.

### Example 5

### Analysis of Alternative Splice Variants in Small Cell Lung Cancer

We have identified alternative splice variants of regulatory genes that are expressed in small cell lung cancer cells and analyzed expression of these splice variants in biopsy of primary small cell lung cancer tumor, lymph node metastasis and circulating tumor cells using RT-PCR technique. Also, we analyzed presence of auto-antibodies in small cell lung cancer patients blood serum. For RT-PCR analyses we used primers located 50-100 bp 5′ and 3′ from the alternative splice junction. To detect auto-antibodies we used synthetic peptides corresponding to unique isoforms generated by alternative splicing. Following markers were used in this study (Table 2).

TABLE 2
Alternative splice variants of regulatory factors and corresponding
peptide sequences that were used to analyze mRNA expression
and presence of auto-antibodies in small cell lung cancer biopsies,
blood samples and serum.

| | | | | | Genebank accession |
|---|---|---|---|---|---|
| # | Gene | Aa | peptide seq | mer | # |
| A1 | NRSF | 133-150 | RTHSVGYGYHLVIFTRV | 17 | AF228045 |
| A2 | MDM2-A | 24-36 | QETLDLDAGVSEH | 13 | NM_006878 |
| A3 | MDM2b | 22-33 | SEQETLDYWKCT | 12 | NM_006879 |
| A4 | MDM2c1 | 50-62 | MKEVLDAGVSEHS | 12 | NM_006880 |
| A5 | MDM2c2 | 25-36 | ETLVRQESEDYS | 12 | NM_006880 |
| A6 | TSG101 | 10-21 | KMVSKFLTMAVP | 12 | AY207474 |
| A7 | RREB-1(1) | 11-24 | AQQASPGCISPQPA | 14 | AI924329 |
| A8 | RREB-1(2) | 1247-1257 | HMLTHTDSQSDAG | 13 | NP_002946 |
| B1 | ZNF207(1) | 41-54 | HKKLYTGLPPVPGA | 14 | AI870134 |
| B2 | TTF-1 (1) | 120-133 | PRFPAISRFMGPAS | 14 | BAA23529 |
| B3 | TTF-1 (2) | 154-168 | APLPTAPGRKRRVLF | 15 | BAA23529 |
| B4 | TTF-1 (3) | 154-166 | APLPSAPRRKRRV | 13 | BC006221 |
| B5 | TTF-1 (4) | 16-28 | AGGRSSPGRLSRR | 13 | BC006221 |
| B6 | TTF-1 (5)N | 212-224 | HRYKMKRQAKDKA | 13 | NM_003317 |
| B7 | TTF-1 (6)N | 315-330 | AHPGHQPGSAGQSPDL | 16 | NM_003317 |
| B8 | GTFIIIA (1) | 292-304 | KRSLASHLSGYIP | 13 | U14134 |
| C1 | GTFIIIA (2) | 375-388 | EKREFGLSSQWIYP | 14 | NP_002088 |
| C2 | HES-6 | 99-113 | VTPARRRTSLPAPLS | 15 | AK075040 |
| C3 | HRY (1) | 12-24 | SPVAASVNTTPDK | 13 | BC039152 |
| C4 | HRY (2) | 12-25 | SPVAATPASVNTTP | 14 | AF264785 |
| C5 | Msx2 (1) | 60-73 | KESPAVPPEGASAG | 14 | X69295 |
| C6 | Msx2 (2) | 60-73 | KEASPLPAESASAG | 14 | D31771 |

RT-PCR Analysis

We compared the expression of alternative splice variants of regulatory factors in primary tumor, lymph node metastasis and circulating cancer cells using RT-PCR analysis (Table 3).

TABLE 3
Expression of mRNA alternative splice variants (ASV)in primary tumor, lymph nodes and
circulating cancer cells using RT-PCR analysis
(+ASV is expressed; −ASV is not detected).

| | Patient 1 | | | Patient 2 | | | Patient 3 | | | Patient 4 | | | Patient 5 | | | Control 1 | | | Control 2 | | |
|---|---|---|---|---|---|---|---|---|---|---|---|---|---|---|---|---|---|---|---|---|---|
| MARKERS | PT | LN | CC | PT | LN | CC | PT | LN | CC | PT | LN | CC | PT | LN | CC | Lung | LN | CC | Lung | LN | CC |
| NRSF | + | + | + | + | + | + | + | + | − | + | − | − | + | + | + | − | − | − | − | − | − |
| MDM2-A | + | + | − | + | + | + | + | + | + | + | + | + | + | + | + | − | − | − | − | − | − |
| MDM2b | + | − | − | + | + | + | + | + | + | + | + | + | + | + | + | − | − | − | − | − | − |
| MDM2c1 | + | + | − | + | + | + | + | + | + | + | + | + | − | − | − | − | − | − | − | − | − |
| MDM2c2 | + | + | + | + | + | + | + | + | + | + | + | + | + | + | + | − | − | − | − | − | − |
| TSG101 | + | + | + | + | + | + | + | + | + | + | + | + | + | + | + | − | − | − | − | − | − |
| RREB-1(1) | + | + | + | − | + | − | + | + | + | + | + | + | + | − | − | − | − | − | − | − | − |
| RREB-1(2) | + | + | + | + | + | − | + | + | − | + | − | − | + | + | − | − | − | − | − | − | − |
| ZNF207(1) | + | − | + | + | + | + | + | + | − | + | + | + | + | − | + | − | − | − | − | − | − |
| TTF-1 (1) | + | + | − | + | + | + | + | + | + | + | + | + | + | + | + | − | − | − | − | − | − |
| TTF-1 (2) | + | + | + | + | + | + | + | + | + | + | + | + | + | + | + | − | − | − | − | − | − |
| TTF-1 (3) | + | + | + | + | + | + | + | − | − | + | + | + | + | + | − | − | − | − | − | − | − |
| TTF-1 (4) | + | + | + | + | + | + | − | − | − | + | + | + | + | + | + | − | − | − | − | − | − |
| TTF-1 (5)N | + | + | + | + | − | − | + | − | − | + | + | + | + | + | + | − | − | − | − | − | − |
| TTF-1 (6)N | + | + | + | + | + | + | + | + | + | + | + | + | + | + | + | − | − | − | − | − | − |
| GTFIIIA (1) | + | + | + | + | + | + | + | + | + | + | + | + | + | + | + | − | − | − | − | − | − |
| GTFIIIA (2) | + | + | + | + | + | + | + | + | + | + | + | + | + | + | + | − | − | − | − | − | − |
| HES-6 | + | + | + | + | + | + | + | + | − | + | + | + | + | + | + | − | − | − | − | − | − |
| HRY (1) | + | + | + | + | + | + | + | + | − | − | − | − | + | + | − | − | − | − | − | − | − |
| HRY (2) | + | − | + | − | − | − | + | + | + | + | + | + | + | + | + | − | − | − | − | − | − |
| Msx2 (1) | + | + | − | − | − | − | + | + | + | + | + | + | + | + | + | − | − | − | − | − | − |
| Msx2 (2) | + | + | − | + | + | + | + | + | + | + | + | + | + | + | + | − | − | − | − | − | − |

Results of RT-PCR analysis show that primary tumor, lymph nodes and blood cells express similar pattern of alternative splice variants whereas control (healthy) patients lung, lymph nodes and blood does not express these alternative splice variants at detectable level. These data also show that individual patients express different sets of alternative splice variants. Difference in the expression of alternative splice variants in cancer, lymph nodes and blood allows us to suggest that these splice variants can be used to detect and diagnose small cell lung cancer.

### Example 6

### Analysis of Auto-Antibodies Against Isoforms of Regulatory Factors Generated by Alternative Splicing

We synthesized antigenic peptides corresponding to alternative splice variants of regulatory factors and used these peptides to analyze presence of antibodies in small cell lung cancer patients blood. Our results clearly demonstrate that small cell lung cancer patients have auto-antibodies against regulatory factor isoforms that carry specific epitopes (Table 4).

TABLE 4
Analysis of auto-antibodies in small cell lung cancer patients
using peptide array.

| | | SCLC | | | | | CONTROL | | | | |
|---|---|---|---|---|---|---|---|---|---|---|---|
| | Marker | 1 | 2 | 3 | 4 | 5 | 1 | 2 | 3 | 4 | 5 |
| A1 | NRSF | 1 | 3 | 1 | 2 | 2 | 0 | 0 | 0 | 0 | 0 |
| A2 | MDM2-A | 2 | 0 | 1 | 0 | 2 | 2 | 0 | 0 | 0 | 1 |
| A3 | MDM2b | 0 | 0 | 0 | 1 | 0 | 0 | 0 | 0 | 0 | 0 |
| A4 | MDM2c1 | 1 | 0 | 0 | 0 | 0 | 0 | 0 | 0 | 0 | 0 |
| A5 | MDM2c2 | 3 | 2 | 3 | 1 | 3 | 0 | 0 | 0 | 0 | 0 |
| A6 | TSG101 | 2 | 1 | 3 | 2 | 2 | 0 | 0 | 0 | 0 | 0 |
| A7 | RREB- | 0 | 0 | 1 | 0 | 0 | 0 | 0 | 0 | 0 | 0 |
| | 1(1) | | | | | | | | | | |
| A8 | RREB- | 1 | 1 | 1 | 1 | 1 | 0 | 0 | 0 | 0 | 0 |
| | 1(2) | | | | | | | | | | |
| B1 | ZNF207(1) | 2 | 3 | 3 | 3 | 3 | 0 | 0 | 0 | 0 | 0 |
| B2 | TTF-1 (1) | 2 | 1 | 2 | 2 | 1 | 0 | 0 | 0 | 0 | 0 |
| B3 | TTF-1 (2) | 0 | 0 | 0 | 0 | 0 | 0 | 0 | 0 | 0 | 0 |
| B4 | TTF-1 (3) | 3 | 3 | 3 | 2 | 3 | 0 | 0 | 0 | 0 | 0 |
| B5 | TTF-1 (4) | 1 | 0 | 0 | 0 | 1 | 0 | 0 | 0 | 0 | 0 |
| B6 | TTF-1 | 0 | 0 | 0 | 1 | 1 | 1 | 1 | 1 | 0 | 0 |
| | (5)N | | | | | | | | | | |
| B7 | TTF-1 | 0 | 0 | 0 | 0 | 0 | 0 | 0 | 0 | 0 | 0 |
| | (6)N | | | | | | | | | | |
| B8 | GTFIIIA | 3 | 1 | 3 | 2 | 2 | 0 | 0 | 0 | 0 | 0 |
| | (1) | | | | | | | | | | |
| C1 | GTFIIIA | 0 | 0 | 1 | 0 | 0 | 0 | 0 | 0 | 0 | 0 |
| | (2) | | | | | | | | | | |
| C2 | HES-6 | 2 | 2 | 1 | 3 | 2 | 0 | 0 | 0 | 0 | 0 |
| C3 | HRY (1) | 1 | 1 | 1 | 1 | 1 | 0 | 0 | 0 | 0 | 0 |
| C4 | HRY (2) | 1 | 0 | 0 | 0 | 0 | 0 | 0 | 0 | 0 | 0 |
| C5 | Msx2 (1) | 0 | 0 | 0 | 1 | 3 | 0 | 0 | 0 | 0 | 0 |
| C6 | Msx2 (2) | 0 | 1 | 0 | 0 | 1 | 0 | 0 | 0 | 0 | 0 |

SCLC 1—patients 1-5

SCLC 2—patients 6-10

SCLC 3—patients 11-15

SCLC 4—patients 16-20

SCLC 5—patients 21-25

Control 1-5—Random Samples, Pooled 5 Patients

0—undetectable

1—dilution 1/10-1/500

2—dilution 1/1000-1/10,000

3—dilution 1/100,000-1/1,000,000

SCLC Study

Analyses of alternative splice variants of transcription factors clearly demonstrated that primary tumor, lymph node metastasis and circulating cancer cells express similar splice variants. From a large number of expressed transcription factors in Small Cell Lung Cancer (SCLC) cells we identified a set of splice variants that are expressed in tumor cells whereas expression in normal lung tissue is undetectable (see Table 1)

Since all these alternative splice variants encode proteins with altered amino acid sequences we synthesized peptides corresponding to isoform specific sequences and used these peptides to study presence of auto-antibodies against these peptide epitopes.

The most informative markers for SCLC detection using auto-antibodies against isoform specific peptides were:

| Marker | Peptide |
|---|---|
| NRSF | RTHSVGYGYHLVIFTRV |
| MDM2A | QETLDLDAGVSEH |
| MDM2C2 | ETLVRQESEDYS |
| TSG101 | KMVSKFLTMAVP |
| PREB1(2) | HMLTHTDSQSDAG |
| ZNF207 | HKKLYTGLPPVPGA |
| TTF1(1) | PRFPAISRFMGPAS |
| TTF1(3) | APLPSAPRRKRRV |
| GTFIIA1 | KRSLASHLSGYIP |
| HES6 | VTPARRRTSLPAPLS |
| HRY(1) | SPVAASVNTTPDK |
| MSX2(1) | KESPAVPPEGASAG |

Primers that were used to analyze alternative splice variants of transcription factor in SCLC primary tumor, lymph node and circulating cancer cells.

| | | |
|---|---|---|
| Gene | Forward primer | Reverse primer |

| | | |
|---|---|---|
| NRSF | 5′-cacctgaaacaccacaccag | 5′-gcccattgtgaacctgtctt |
| MDM2-A | 5′-gagcaggcaaatgtgcaata | 5′-tctgagagttcttgtccttcttca |
| MDM2b | 5′-gagcaggcaaatgtgcaata | 5′-tgttgcaatgtgatggaagg |
| MDM2c1 | 5′-gaccctggttagaccaaagc | 5′-cctgatccaaccaatcacct |
| MDM2c2 | 5′-gagcaggcaaatgtgcaata | 5′-tttttgtgcaccaacagacttt |
| TSG101 | 5′-gagccagctcaagaaaatgg | 5′-gacctgaataagccccaaca |
| RREB-1 (1) | 5′-cgcgctgctactcacatact | 5′-caaccaggtgtttgccttct |
| RREB-1 (2) | 5′-gtgatgaagagcagggcagt | 5′-gtcccgtgaggtgaggtcta |
| ZNF207 (1) | 5′-agttcctggtatgtgggaaga | 5′-tcctgtaatgtcgcaaggt |
| TTF-1 (1) | 5′-aggacaccatgaggaacagc | 5′-gccatgttcttgctcacgtc |
| TTF-1 (2) | 5′-accaggacaccatgaggaac | 5′-gggccatgttcttgctcac |
| TTF-1 (3) | 5′-gagcggcatgaacatgag | 5′-gtcgctccagctcgtacac |
| TTF-1 (4) | 5′-gccgaatcatgtcgatgag | 5′-ccctccatgcccactttct |
| TTF-1 (5)N | 5′-ccagcatgatccacctgac | 5′-gctgagcctgttgctgct |
| TTF-1 (6)N | 5′-caacaggctcagcagcagt | 5′-gaggagttcaggtgggacag |
| GTFIIIA (1) | 5′-aaaaacggagtttggcctct | 5′-ctgcaactgtcgagagcatc |
| GTFIIIA (2) | 5′-ggcaaaacatttgcaatgaa | 5′-cttgcccttgtttccttttg |
| HES-6 | 5′-ccgaagtgctggagctgac | 5′-gagggtgggagggagaga |
| HRY (1) | 5′-aaaaggaaaatgccagctgat | 5′-tgctcttcgtcttttctcca |
| HRY (2) | 5′-aaattcctcgtccccggtag | 5′-tcagctggctcagactttca |
| Msx2 (1) | 5′-gtctccagcctgcccttc | 5′-ccgattggtcttgtgtttcc |
| Msx2 (2) | 5′-gtctccagcctgcccttc | 5′-ctgaatttcccgacttgacc |

### Example 7

### Alternative Splice Variant Profiling for Early Detection of Cancer

The example discussed below concerns a population at high risk for cancer, particularly heavy smokers. However, the present methods may be used with any high risk group, for example those with a familial history of cancer. A person may fall within a high risk group because behavioral, and/or genetic, and/or environmental factors suggest that they are at high risk for cancer.

Analysis of Heavy Smokers for the Presence of Auto-Antibodies Against Protein Isoforms Encoded by Alternatively Spliced mRNAs

The overall goal of this study is to analyze the presence of auto-antibodies against alternative splice variants of transcription modulators in the high risk group patients blood and validate the early detection technique of lung cancer that is based on the identification of a set of auto-antibodies. Blood is collected from 1200 high risk group individuals and from 100 lung cancer patients (positive control). The presence of auto-antibodies to 120 splice variants of transcription modulators are simultaneously analyzed using an array technique. Differences in the auto-antibody profile between normal and high risk group individuals are observed. Auto-antibody patterns similar to those of lung cancer patients are observed in a significant number of patients, and indicate the presence of lung cancer in these high risk group individuals.

Experimental Design and Protocols of the Study

(I) Selection of Individuals for the Study

Two groups of individuals

I. High Risk Group, Heavy Smokers

Individuals who will be selected for this study should correspond to the following criteria

1. long time smokers—20-25 years, more than 20 cigarettes a day

2. age—over 50

3. no diagnosed lung or any other tumor

II. Patients with Diagnosed Lung Cancer

Collection of Blood and Preparation of Serum

5 ml blood will be collected from each individual by venipuncture into EDTA tubes and used to prepare blood plasma. Plasma will be prepared immediately after blood drawing. 5 ml of blood will be centrifuged at 170×g for 5 minutes after which plasma is removed.

Plasma will be aliquoted and stored at −20 C.

Analyses of Auto-Antibody Profile

Analyses Technique: Array Analyses with Immunogenic Peptides.

1) Peptide: 1 mg/ml in H₂O

Print a peptide array on nitrocellulose covered microscope glass (Schleicher@Schuell)

Air dry array following printing

Blocking overnight in blocking solution at +4° C.

Blocking solution: PBS; 0.1% Tween 20; 1% casein; 1% goat serum; 5 mM EDTA

2) Incubate array with test serum, dilution 1:50. Dilution is made into blocking solution.

3) Wash 4 times with PBS, 0.1% Tween 20

4) Incubate with secondary antibody: goat anti human Ig conjugated to peroxidase) or alkaline phosphatase or fluorescent label) dilution in blocking solution (1:1000, Dako)

5) Wash 4 times with PBS, 0.1% Tween 20

6) Color reaction for peroxidase

Substrate:

Stock solutions: diaminobenzidine (DAB, Sigma -D-5637)-10 mg in 5 ml methanol

Chloronaphthol 30 mg in 5 ml methanol

Working solution, make fresh: 0.5 ml of DAB stock +0.5 ml chloronaphtole stock +4 ml PBS+5 microliters of H₂O₂

7) Densitometry scan of microarrays.

1. A method for diagnosing cancer, comprising determining the expression of at least one splice variant of each of a plurality of transcription modulators, wherein expression of each of said splice variants is distinguished from expression of the wildtype isoform of the corresponding transcription modulator, and wherein the expression pattern of said splice variants is indicative of cancer.

2. The method according to claim 1, further comprising determining the expression of a plurality of splice variants of at least one of said plurality of transcription modulators, wherein expression of each of said splice variants is distinguished from expression of the wildtype isoform of the corresponding transcription modulator, and wherein the expression pattern of said splice variants is indicative of cancer.

3. A method for diagnosing cancer, comprising determining the expression of a plurality of splice variants of at least one transcription modulator, wherein expression of each of said splice variants is distinguished from expression of the wildtype isoform of the corresponding transcription modulator, and wherein the expression pattern of said splice variants is indicative of cancer.

4. The method according to claim 3, further comprising determining the expression of a plurality of splice variants of a plurality of transcription modulators, wherein expression of each of said splice variants is distinguished from expression of the wildtype isoform of the corresponding transcription modulator, and wherein the expression pattern of said splice variants is indicative of cancer.

5. The method according to any one of claims 1 to 4, wherein the expression pattern of said splice variants is indicative of at least one cancer selected from the group consisting of lung cancer, gastrointestinal cancer, breast cancer, prostate cancer, skin cancer, sarcoma, endocrine cancer, neural cancer, bladder cancer, cervical cancer, renal cancer, and hematopoietic cancer.

6. The method according to claim 1, 2, or 4, wherein one or more of said transcription modulators is selected from the group consisting of NRSF, MDM2 TSG, RREB, ZNF207, TTF-1, GTFIIIA, HES-6, HRY, Msx2, Neu, NeuroD1, Mash-1, and Irx2.

7. The method according to claim 3, wherein said at least one transcription modulator is selected from the group consisting of NRSF, MDM2 TSG, RREB, ZNF207, TTF-1, GTFIIIA, HES-6, HRY, Msx2, Neu, NeuroD1, Mash-1, and Irx2.

8. The method according to any one of claims 1, 2, or 4, wherein one or more said splice variants is selected from the group consisting of the sequences set forth at Genbank accession numbers AF228045, NM_{—}006878, NM_{—}006879, NM_{—}006880, NM_{—}006880, AY207474, AI924329, NP_{—}002946, AI870134, BAA23529, BAA23529, BC006221, BC006221, NM_{—}003317, NM_{—}003317, U14134, NP_{—}002088, AK075040, BC039152, AF264785, X69295, D31771, and the splice variant peptide sequences listed in FIGS. 4 to 7 (SEQ ID NOS:27-42).

9. The method according to claim 3, wherein one of said plurality of splice variants is selected from the group consisting of the sequences set forth at Genbank accession numbers AF228045, NM_{—}006878, NM_{—}006879, NM_{—}006880, NM_{—}006880, AY207474, AI924329, NP_{—}002946, AI870134, BAA23529, BAA23529, BC006221, BC006221, NM_{—}003317, NM_{—}003317, U14134, NP_{—}002088, AK075040, BC039152, AF264785, X69295, D31771, and the splice variant peptide sequences listed in FIGS. 4 to 7 (SEQ ID NOS:27-42).

10. The method according to any one of claims 1 to 4, wherein the expression pattern of said splice variants is determined simultaneously.

11. The method according to any one of claims 1 to 4, wherein said determining the expression of at least one splice variant comprises determining the expression of at least one mRNA encoding said at least one splice variant.

12. The method according to claim 11, wherein said determining the expression of at least one mRNA comprises the use of a nucleic acid array.

13. The method according to claim 11, wherein said determining the expression of at least one mRNA comprises the use of RT-PCR.

14. The method according to any one of claims 1 to 4, wherein said determining the expression of at least one splice variant comprises determining the presence of an autoantibody in a sample, which autoantibody specifically binds to said at least one splice variant.

15. The method according to claim 14, wherein said determining the presence of an autoantibody comprises the use of a peptide that specifically binds to said autoantibody.

16. The method according to claim 15, wherein said peptide comprises an amino acid sequence selected from the group consisting of

| | |
|---|---|
| RTHSVGYGYHLVIFTRV, | (SEQ ID NO: 1) |
| QETLDLDAGVSEH, | (SEQ ID NO: 2) |
| SEQETLDYWKCT, | (SEQ ID NO: 3) |
| MKEVLDAGVSEHS, | (SEQ ID NO: 4) |
| ETLVRQESEDYS, | (SEQ ID NO: 5) |
| KMVSKFLTMAVP, | (SEQ ID NO: 6) |
| SPGCISPQPA, | (SEQ ID NO: 7) |
| HMLTHTDSQSDAG, | (SEQ ID NO: 8) |
| HKKLYTGLPPVPGA, | (SEQ ID NO: 9) |
| PRFPAISRFMGPAS, | (SEQ ID NO: 10) |
| APLPTAPGRKRRVLF, | (SEQ ID NO: 11) |
| APLPSAPRRKRRV, | (SEQ ID NO: 12) |
| AGGRSSPGRLSRR, | (SEQ ID NO: 13) |
| HRYKMKRQAKDKA, | (SEQ ID NO: 14) |
| AHPGHQPGSAGQSPDL, | (SEQ ID NO: 15) |
| KRSLASHLSGYIP, | (SEQ ID NO: 16) |
| EKREFGLSSQWIYP, | (SEQ ID NO: 17) |
| VTPARRRTSLPAPLS, | (SEQ ID NO: 18) |
| SPVAASVNTTPDK, | (SEQ ID NO: 19) |
| SPVAATPASVNTTP, | (SEQ ID NO: 20) |
| KESPAVPPEGASAG, | (SEQ ID NO: 21) |
| KEASPLPAESASAG, | (SEQ ID NO: 22) |
| GHPQNLKDSELV, | (SEQ ID NO: 23) |
| MNAEEBSLRNGG, | (SEQ ID NO: 24) |
| MRCKRRLNSSGF, and | (SEQ ID NO: 25) |
| CKRLLFRRMYDR. | (SEQ ID NO: 26) |

17. The method according to claim 16, wherein said peptide consists essentially of an amino acid sequence selected from the group consisting of

| | |
|---|---|
| RTHSVGYGYHLVIFTRV, | (SEQ ID NO: 1) |
| QETLDLDAGVSEH, | (SEQ ID NO: 2) |
| SEQETLDYWKCT, | (SEQ ID NO: 3) |
| MKEVLDAGVSEHS, | (SEQ ID NO: 4) |
| ETLVRQESEDYS, | (SEQ ID NO: 5) |
| KMVSKFLTMAVP, | (SEQ ID NO: 6) |
| SPGCISPQPA, | (SEQ ID NO: 7) |
| HMLTHTDSQSDAG, | (SEQ ID NO: 8) |
| HKKLYTGLPPVPGA, | (SEQ ID NO: 9) |
| PRFPAISRFMGPAS, | (SEQ ID NO: 10) |
| APLPTAPGRKRRVLF, | (SEQ ID NO: 11) |
| APLPSAPRRKRRV, | (SEQ ID NO: 12) |
| AGGRSSPGRLSRR, | (SEQ ID NO: 13) |
| HRYKMKRQAKDKA, | (SEQ ID NO: 14) |
| AHPGHQPGSAGQSPDL, | (SEQ ID NO: 15) |
| KRSLASHLSGYIP, | (SEQ ID NO: 16) |
| EKREFGLSSQWIYP, | (SEQ ID NO: 17) |
| VTPARRRTSLPAPLS, | (SEQ ID NO: 18) |
| SPVAASVNTTPDK, | (SEQ ID NO: 19) |
| SPVAATPASVNTTP, | (SEQ ID NO: 20) |
| KESPAVPPEGASAG, | (SEQ ID NO: 21) |
| KEASPLPAESASAG, | (SEQ ID NO: 22) |
| GHPQNLKDSELV, | (SEQ ID NO: 23) |
| MNAEEBSLRNGG, | (SEQ ID NO: 24) |
| MRCKRRLNSSGF, and | (SEQ ID NO: 25) |
| CKRLLFRRMYDR. | (SEQ ID NO: 26) |

18. The method according to any one of claims 15-17, further comprising the use of a peptide array.

19. The method according to any one of claims 1 to 4, wherein the expression pattern of said splice variants is indicative of a cancer subtype.

20. A method for determining cancer prognosis, comprising determining the expression of at least one splice variant of each of a plurality of transcription modulators, wherein the expression pattern of said splice variants is indicative of a cancer subtype.

21. A method for determining cancer prognosis, comprising determining the expression of a plurality of splice variants of at least one transcription modulator, wherein the expression pattern of said splice variants is indicative of a cancer subtype.

22. A method for the treatment of cancer, comprising:
a) determining the expression of at least one splice variant of each of a plurality of transcription modulators;
b) administering to said patient a bioactive agent capable of inhibiting the activity of one or more of said splice variants;
wherein expression of each splice variant is distinguished from expression of the wildtype isoform of the corresponding transcription modulator, and wherein the expression pattern said splice variants is indicative of cancer.

23. A method for the treatment of cancer, comprising:
a) determining the expression of a plurality of splice variants of at least one transcription modulator;
b) administering to said patient a bioactive agent capable of inhibiting the activity of one or more of said splice variants;
wherein expression of each splice variant is distinguished from expression of the wildtype isoform of the corresponding transcription modulator, and wherein the expression pattern of said splice variants is indicative of cancer.

24. The method according to claim 22 or 23, wherein the expression pattern of said splice variants is indicative of at least one cancer selected from the group consisting of lung cancer, gastrointestinal cancer, breast cancer, prostate cancer, skin cancer, sarcoma, endocrine cancer, neural cancer, bladder cancer, cervical cancer, renal cancer, and hematopoietic cancer.

25. The method according to claim 22, wherein one or more of said plurality of transcription modulators is selected from the group consisting of NRSF, MDM2 TSG, RREB, ZNF207, TTF-1, GTFIIIA, HES-6, HRY, Msx2, Neu, NeuroD1, Mash-1, and Irx2.

26. The method according to claim 23, wherein said transcription modulator is selected from the group consisting of NRSF, MDM2 TSG, RREB, ZNF207, TTF-1, GTFIIIA, HES-6, HRY, Msx2, Neu, NeuroD1, Mash-1, and Irx2.

27. The method according to claim 22, wherein one or more said splice variants is selected from the group consisting of the sequences set forth at Genbank accession numbers AF228045, NM 006878, NM 006879, NM_{—}006880, NM_{—}006880, AY207474, AI924329, NP_{—}002946, AI870134, BAA23529, BAA23529, BC006221, BC006221, NM_{—}003317, NM_{—}003317, U14134, NP_{—}002088, AK075040, BC039152, AF264785, X69295, D31771, and the splice variants disclosed in FIGS. 4 to 7 (SEQ ID NOS:27-42).

28. The method according to claim 23, wherein one of said plurality of splice variants is selected from the group consisting of the sequences set forth at Genbank accession numbers AF228045, NM_{—}006878, NM_{—}006879, NM_{—}006880, NM_{—}006880, AY207474, AI924329, NP_{—}002946, AI870134, BAA23529, BAA23529, BC006221, BC006221, NM_{—}003317, NM_{—}003317, U14134, NP_{—}002088, AK075040, BC039152, AF264785, X69295, D31771, and the splice variants disclosed in FIGS. 4 to 7 (SEQ ID NOS:27-42).

29. The method according to claim 22 or 23, wherein said bioactive agent is a small interfering RNA.

30. The method according to claim 22 or 23, wherein said bioactive agent is an antisense nucleic acid.

31. The method according to claim 22 or 23, wherein said bioactive agent is a small molecule chemical compound.

32. The method according to claim 22 or 23, wherein said bioactive agent is a decoy oligonucleotide which is capable of binding to said at least one splice variant of a first transcription modulator.

33. The method according to claim 22 or 23, wherein said bioactive active agent directly targets one or more of said splice variants and is selective for said one or more splice variants over the wildtype isoforms of said the corresponding one or more transcription modulators.

34. The method according to claim 22 or 23, wherein determining the expression of said splice variants comprises determining the expression of at least one mRNA encoding at least one of said splice variants.

35. The method according to claim 34, wherein said determining the expression of at least one mRNA comprises the use of a nucleic acid array.

36. The method according to claim 34, wherein said determining the expression of at least one mRNA comprises the use of RT-PCR.

37. The method according to claim 22 or 23, wherein determining the expression of said splice variants comprises determining the presence of an autoantibody in a sample, which autoantibody specifically binds to said at least one splice variant.

38. The method according to claim 37, wherein said determining the presence of an autoantibody comprises the use of a peptide that specifically binds to said autoantibody.

39. The method according to claim 38, wherein said peptide comprises an amino acid sequence selected from the group consisting of

| | |
|---|---|
| RTHSVGYGYHLVIFTRV, | (SEQ ID NO: 1) |
| QETLDLDAGVSEH, | (SEQ ID NO: 2) |
| SEQETLDYWKCT, | (SEQ ID NO: 3) |
| MKEVLDAGVSEHS, | (SEQ ID NO: 4) |
| ETLVRQESEDYS, | (SEQ ID NO: 5) |
| KMVSKFLTMAVP, | (SEQ ID NO: 6) |
| SPGCISPQPA, | (SEQ ID NO: 7) |
| HMLTHTDSQSDAG, | (SEQ ID NO: 8) |
| HKKLYTGLPPVPGA, | (SEQ ID NO: 9) |
| PRFPAISRFMGPAS, | (SEQ ID NO: 10) |
| APLPTAPGRKRRVLF, | (SEQ ID NO: 11) |
| APLPSAPRRKRRV, | (SEQ ID NO: 12) |
| AGGRSSPGRLSRR, | (SEQ ID NO: 13) |
| HRYKMKRQAKDKA, | (SEQ ID NO: 14) |
| AHPGHQPGSAGQSPDL, | (SEQ ID NO: 15) |
| KRSLASHLSGYIP, | (SEQ ID NO: 16) |
| EKREFGLSSQWIYP, | (SEQ ID NO: 17) |
| VTPARRRTSLPAPLS, | (SEQ ID NO: 18) |
| SPVAASVNTTPDK, | (SEQ ID NO: 19) |
| SPVAATPASVNTTP, | (SEQ ID NO: 20) |
| KESPAVPPEGASAG, | (SEQ ID NO: 21) |
| KEASPLPAESASAG, | (SEQ ID NO: 22) |
| GHPQNLKDSELV, | (SEQ ID NO: 23) |
| MNAEEBSLRNGG, | (SEQ ID NO: 24) |
| MRCKRRLNSSGF, and | (SEQ ID NO: 25) |
| CKRLLFRRMYDR. | (SEQ ID NO: 26) |

40. The method according to claim 39, wherein said peptide consists essentially of an amino acid sequence selected from the group consisting of

| | |
|---|---|
| RTHSVGYGYHLVIFTRV, | (SEQ ID NO: 1) |
| QETLDLDAGVSEH, | (SEQ ID NO: 2) |
| SEQETLDYWKCT, | (SEQ ID NO: 3) |
| MKEVLDAGVSEHS, | (SEQ ID NO: 4) |
| ETLVRQESEDYS, | (SEQ ID NO: 5) |
| KMVSKFLTMAVP, | (SEQ ID NO: 6) |
| SPGCISPQPA, | (SEQ ID NO: 7) |
| HMLTHTDSQSDAG, | (SEQ ID NO: 8) |
| HKKLYTGLPPVPGA, | (SEQ ID NO: 9) |
| PRFPAISRFMGPAS, | (SEQ ID NO: 10) |
| APLPTAPGRKRRVLF, | (SEQ ID NO: 11) |
| APLPSAPRRKRRV, | (SEQ ID NO: 12) |
| AGGRSSPGRLSRR, | (SEQ ID NO: 13) |
| HRYKMKRQAKDKA, | (SEQ ID NO: 14) |
| AHPGHQPGSAGQSPDL, | (SEQ ID NO: 15) |
| KRSLASHLSGYIP, | (SEQ ID NO: 16) |
| EKREFGLSSQWIYP, | (SEQ ID NO: 17) |
| VTPARRRTSLPAPLS, | (SEQ ID NO: 18) |
| SPVAASVNTTPDK, | (SEQ ID NO: 19) |
| SPVAATPASVNTTP, | (SEQ ID NO: 20) |
| KESPAVPPEGASAG, | (SEQ ID NO: 21) |
| KEASPLPAESASAG, | (SEQ ID NO: 22) |
| GHPQNLKDSELV, | (SEQ ID NO: 23) |
| MNAEEBSLRNGG, | (SEQ ID NO: 24) |
| MRCKRRLNSSGF, and | (SEQ ID NO: 25) |
| CKRLLFRRMYDR. | (SEQ ID NO: 26) |

41. The method according to any of claims 38 to 40, further comprising the use of a peptide array.

42. The method according to claim 22 or 23, wherein the expression pattern of said splice variants is indicative of a cancer subtype.

43. A nucleic acid encoding a transcription modulator splice variant, comprising the nucleotide sequence set forth in FIG. 4 (SEQ ID NOS:27-30).

44. A transcription modulator splice variant, comprising an amino acid sequence encoded by the nucleic acid of claim 43.

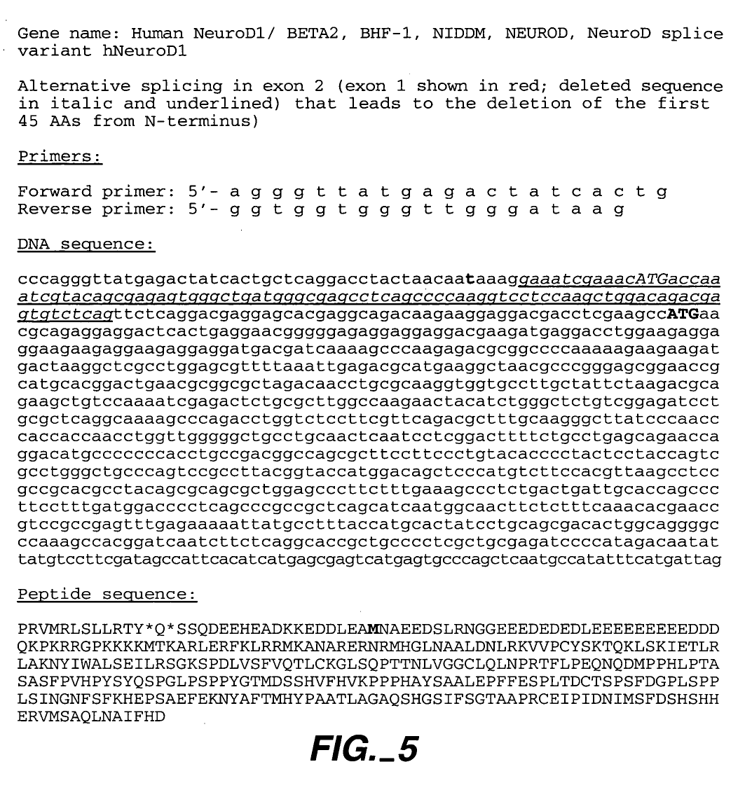

45. A nucleic acid encoding a transcription modulator splice variant, comprising the nucleotide sequence set forth in FIG. 5 (SEQ ID NOS:31-35).

46. A transcription modulator splice variant, comprising an amino acid sequence encoded by the nucleic acid of claim 45.

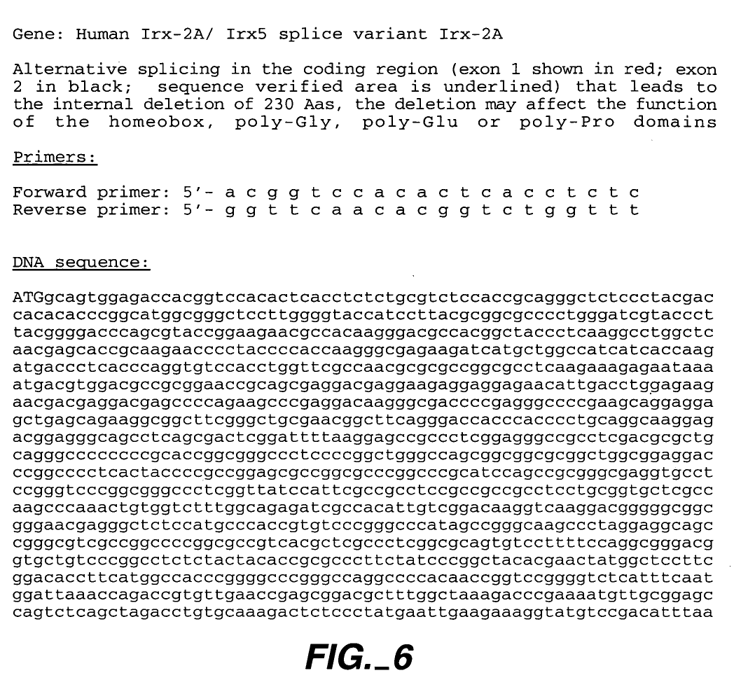

47. A nucleic acid encoding a transcription modulator splice variant, comprising the nucleotide sequence set forth in FIG. 6 (SEQ ID NOS:36-38).

48. A transcription modulator splice variant, comprising an amino acid sequence encoded by the nucleic acid of claim 47.

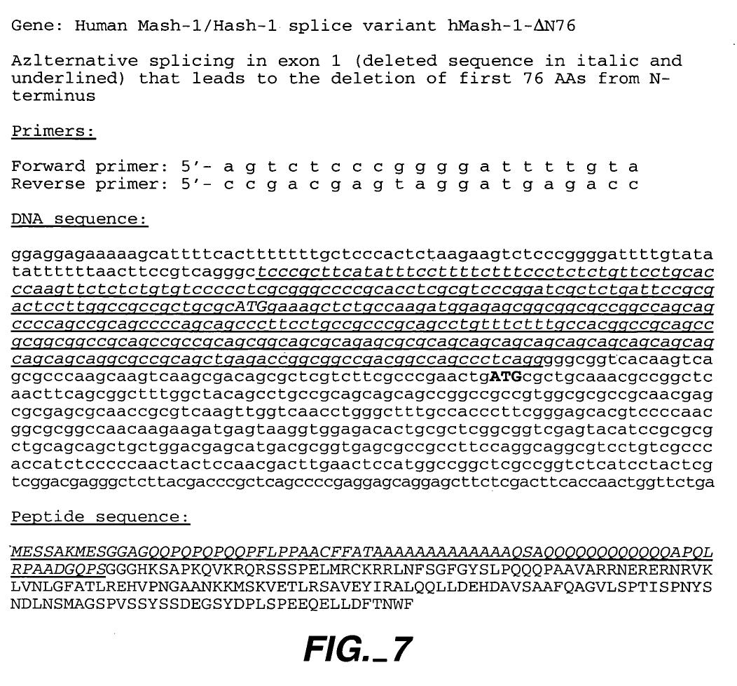

49. A nucleic acid encoding a transcription modulator splice variant, comprising the nucleotide sequence set forth in FIG. 7 (SEQ ID NOS:39-42).

50. A transcription modulator splice variant, comprising an amino acid sequence encoded by the nucleic acid of claim 49.

51. An antibody that specifically binds to a transcription modulator splice variant selected from the group consisting of the transcription modulator splice variants of claims 44, 46, 48, and 50.

52. A peptide that specifically binds to an antibody, which antibody specifically binds to a transcription modulator splice variant selected from the group consisting of the transcription modulator splice variants of claims 44, 46, 38, and 50, wherein said peptide does not specifically bind to the wildtype isoform of the corresponding transcription modulator.

53. The peptide according to claim 52, comprising an amino acid sequence selected from the group consisting of GHPQNLKDSELV (SEQ ID NO:23, MNAEEBSLRNGG (SEQ ID NO:24), MRCKRRLNSSGF (SEQ ID NO 25), and CKRLLFRRMYDR (SEQ ID NO:26).

54. The peptide according to claim 52, consisting essentially of an amino acid sequence selected from the group consisting of GHPQNLKDSELV (SEQ ID NO:23), MNAEEBSLRNGG (SEQ ID NO:24), MRCKRRLNSSGF (SEQ ID NO:25), and CKRLLFRRMYDR (SEQ ID NO:26).

55. A diagnostic array for detecting cancer, comprising at least a first peptide capable of binding with an autoantibody that recognizes a splice variant of a first transcription modulator and a second peptide capable of binding with an autoantibody that recognizes a splice variant of a second transcription modulator; wherein said first and second peptides do not specifically bind to autoantibodies that recognize the wildtype isoforms of said first and second transcription modulators.

56. A diagnostic array for detecting cancer, comprising at least a first peptide capable of binding with an autoantibody that recognizes a first splice variant of a transcription modulator and a second peptide capable of binding with an autoantibody that recognizes a second splice variant of said transcription modulator; wherein said first and second peptides do not specifically bind to autoantibodies that recognize the wildtype isoform of said transcription modulator.

57. The array according to claim 55 or 56, wherein said peptides are non-diffusably bound to a solid support.
